SEQUENCE LISTING (73 sequences)

SEQ ID NO: 1 - protein, 24 aa, Homo sapiens
VLGLRMLFAKTVAVNFLLTAKLFF

SEQ ID NO: 2 - protein, 23 aa, Homo sapiens
VIGFRILLLKVAGFNLLMTLRLW

SEQ ID NO: 3 - protein, 22 aa, Homo sapiens
ILLGKATLYAVLVSALVLMAMV

SEQ ID NO: 4 - protein, 23 aa, Homo sapiens
YYMYLLLLLKSVVYFAIITCCLL

SEQ ID NO: 5 - protein, 23 aa, Homo sapiens
YYTYLLLLLKSVVYFAIITCCLL

SEQ ID NO: 6 - protein, 19 aa, Homo sapiens
LLRMGLAFLVLVALVWFLV

SEQ ID NO: 7 - protein, 21 aa, Homo sapiens
LVPVFCGLLVAKSLVLSALLV

SEQ ID NO: 8 - protein, 21 aa, Homo sapiens
AGTVLLLRAGFYAVSFLSVAV

SEQ ID NO: 9 - protein, 21 aa, Homo sapiens
AVIRYSVAIILFTILPFFLLH

SEQ ID NO: 10 - protein, 21 aa, Homo sapiens
VLGIICIVLMATVLKTIVLIP

SEQ ID NO: 11 - protein, 23 aa, Homo sapiens
LTAEVLGIICIVLMATVLKTIVL

SEQ ID NO: 12 - protein, 21 aa, Homo sapiens
PFFFCCFIAVAMGIRFIIMVA

SEQ ID NO: 13 - protein, 23 aa, Homo sapiens
VLGIICIVLMATVLKTIVLIPFL

SEQ ID NO: 14 - protein, 19 aa, Homo sapiens
VLIGTSVVKIPFTILLFFL

SEQ ID NO: 15 - protein, 20 aa, Homo sapiens
VLIGTSVVKIPFTILLFFLL

SEQ ID NO: 16 - protein, 19 aa, Homo sapiens
VLIGTSVVKLPFTILLFFL

SEQ ID NO: 17 - protein, 20 aa, Homo sapiens
ILIGTSVVKIPFTILLFFLL

SEQ ID NO: 18 - protein, 21 aa, Homo sapiens
IVILLAGGFLSKSLVFSVLFA

SEQ ID NO: 19 - protein, 21 aa, Homo sapiens
ILLLLACIFLIKILAASALWA

SEQ ID NO: 20 - protein, 21 aa, Homo sapiens
GNLVRICLGAVILIILAGFLA

SEQ ID NO: 21 - protein, 21 aa, Homo sapiens
GIIVTDVIATLLLALGVFCFA

SEQ ID NO: 22 - protein, 25 aa, Homo sapiens
VSVATIVIVDICITGGLLLLVYYWS

SEQ ID NO: 23 - protein, 21 aa, Homo sapiens
GFLFAEIVSIFVLAVGVYFIA

SEQ ID NO: 24 - protein, 21 aa, Homo sapiens
LCYLLDGILFIYGVILTALFL

SEQ ID NO: 25 - protein, 21 aa, Homo sapiens
LLAGLVAADAVASLLIVGAVF

SEQ ID NO: 26 - protein, 23 aa, Homo sapiens
VLAGIVMGDLVLTVLIALAVYFL

SEQ ID NO: 27 - protein, 22 aa, Homo sapiens
IITAEGIILLFCAVVPGTLLLF

SEQ ID NO: 28 - protein, 21 aa, Homo sapiens
LCYILDAILFLYGIVLTLLYC

SEQ ID NO: 29 - protein, 19 aa, Homo sapiens
FFIPLLVVILFAVDTGLFI

SEQ ID NO: 30 - protein, 21 aa, Homo sapiens
VSFCLVMVLLFAVDTGLYFSV

SEQ ID NO: 31 - protein, 21 aa, Homo sapiens
GVIEGLLSTLGPATVALLFCY

SEQ ID NO: 32 - protein, 20 aa, Homo sapiens
VLIGTSVVLLPFKILLFFLL

SEQ ID NO: 33 - protein, 20 aa, Homo sapiens
VLIILLVGTSVVKLLLFFLL

SEQ ID NO: 34 - protein, 20 aa, Homo sapiens
VLIGTSVVTLPFKILLFFLL

SEQ ID NO: 35 - protein, 20 aa, Homo sapiens
VLILLLLLLLLLKLLLFFLL

SEQ ID NO: 36 - protein, 20 aa, Homo sapiens
VLILLLLGLLLLKLLLFFLL

SEQ ID NO: 37 - protein, 20 aa, Homo sapiens
VLILLLLLALLLKLLLFFLL

SEQ ID NO: 38 - protein, 20 aa, Homo sapiens
VLILLLLLTLLLKLLLFFLL

SEQ ID NO: 39 - protein, 23 aa, Homo sapiens
VLAGIVMGALVLDVLITLAVYFL

SEQ ID NO: 40 - protein, 23 aa, Homo sapiens
VLALAVLGIVMGDVLITLAVYFL

SEQ ID NO: 41 - protein, 23 aa, Homo sapiens
VLAGDVMGTLVLIVLIALAVYFL

SEQ ID NO: 42 - protein, 24 aa, Homo sapiens
GVLAGIVMGDLVLTVLIALAVYFL

SEQ ID NO: 43 - protein, 24 aa, Homo sapiens
GVLAGDVMGTLVLIVLIALAVYFL

SEQ ID NO: 44 - protein, 24 aa, Homo sapiens
GVLAGIVMGALVLDVLITLAVYFL

SEQ ID NO: 45 - protein, 24 aa, Homo sapiens
GVLALAVLGIVMGDVLITLAVYFL

SEQ ID NO: 46 - protein, 24 aa, Homo sapiens
VLGIICIVLMATVLKTIVLIPFLE

SEQ ID NO: 47 - protein, 24 aa, Homo sapiens
VLGIICIVLMATVLLTIVKIPFLE

SEQ ID NO: 48 - protein, 24 aa, Homo sapiens
VLGIICIVLMKTVLATIVLIPFLE

SEQ ID NO: 49 - protein, 24 aa, Homo sapiens
HLHVLIGTSVVKLPFTILLFFLLH

SEQ ID NO: 50 - protein, 24 aa, Homo sapiens
HLHVLIGKSVVTLPFTILLFFLLH

SEQ ID NO: 51 - protein, 24 aa, Homo sapiens
HLHVLIGTSVVTLPFKILLFFLLH

SEQ ID NO: 52 - protein, 24 aa, Homo sapiens
HLHVLILLLLLLLLLKLLLFFLLH

SEQ ID NO: 53 - protein, 20 aa, Homo sapiens
VLIGTSVVLLPFKILLFFLL

SEQ ID NO: 54 - protein, 20 aa, Homo sapiens
VLIGKSVVLLPFTILLFFLL

SEQ ID NO: 55 - protein, 20 aa, Homo sapiens
VLILLLLLKLLLLLLLFFLL

SEQ ID NO: 56 - protein, 20 aa, Homo sapiens
VLILLLLLLLLLKLLLFFLL

SEQ ID NO: 57 - protein, 20 aa, Homo sapiens
VLILKLLLLLLLLLLLFFLL

SEQ ID NO: 58 - protein, 20 aa, Homo sapiens
VLIILLVGTSVVKLLLFFLL

SEQ ID NO: 59 - protein, 20 aa, Homo sapiens
VLIGTSVVTLPFKILLFFLL

SEQ ID NO: 60 - protein, 20 aa, Homo sapiens
VLILLLLLKLLLKLLLFFLL

SEQ ID NO: 61 - protein, 20 aa, Homo sapiens
VLILLLLGKLLLKLLLFFLL

SEQ ID NO: 62 - protein, 20 aa, Homo sapiens
VLILLLLGLLLLKLLLFFLL

SEQ ID NO: 63 - protein, 20 aa, Homo sapiens
VLILLLLLALLLKLLLFFLL

SEQ ID NO: 64 - protein, 20 aa, Homo sapiens
VLILLLLLTLLLKLLLFFLL

SEQ ID NO: 65 - protein, 263 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 66 - protein, 44 aa, Homo sapiens
TTTPAPRPPTPAPTIASQPLSLRPEACRPAAGGAVHTRGLDFAC

SEQ ID NO: 67 - protein, 220 aa, Homo sapiens
RRKPSLLAHPGRLVKSEETVILQCWSDVMFEHFLLHREGTFNDTLRLIGEHIDGVSKANFSIGRMRQDLAGTYRCYGSVPHSPYQFSAPSDPLDIVITGLYEKPSLSAQPGPTVLAGESVTLSCSSWSSYDMYHLSTEGEAHERRFSAGPKVNGTFQADFPLGPATQGGTYRCFGSFHDSPYEWSKSSDPLLVSVTGNPSNSWPSPTEPSSKTGNPRHLH

SEQ ID NO: 68 - protein, 42 aa, Homo sapiens
HRSKRSRLLHSDYMNMTPRRPGPTRKHYQPYAPPRDFAAYRS

SEQ ID NO: 69 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 70 - protein, 49 aa, Homo sapiens
GRLVPRGRGAAEAATRKQRITETESPYQELQGQRSDVYSDLNTQRPYYK

SEQ ID NO: 71 - protein, 189 aa, Homo sapiens
RSKRSRLLHSDYMNMTPRRPGPTRKHYQPYAPPRDFAAYRSSGLRPVQAQAQSDCSCSTVSPGVLAGSRLAVYFLGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 72 - protein, 331 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLY

SEQ ID NO: 73 - protein, 20 aa, Homo sapiens
VLIGTSVVKLPFTILLFFLL

(19) United States
(12) Patent Application Publication
(10) Pub. No.: US 2023/0272068 A1
(43) Pub. Date: Aug. 31, 2023

(54) MODULAR ASSEMBLY RECEPTORS AND USES THEREOF

(71) Applicant: 12343096 CANADA INC., Montreal (CA)

(72) Inventor: Étienne GAGNON, Montreal (CA)

(21) Appl. No.: 17/680,468

(22) PCT Filed: Aug. 20, 2021
(86) PCT No.: PCT/CA2021/051156

§ 371 Date: Feb. 25, 2022

Related U.S. Application Data
(60) Provisional application No. 63/068,760, filed on Aug. 21, 2020.

(51) Int. Cl. C07K 16/28; C07K 14/705; C07K 14/725; C12N 5/0783

(52) U.S. Cl. CPC C07K 16/2803; C07K 14/705; C07K 14/70578; C07K 14/70521; C07K 14/7051; C12N 5/0636; C07K 2317/622; C07K 2319/00

(57) ABSTRACT

The present application relates to modular chimeric receptors, such as chimeric antigen receptors (CARs) comprising chimeric receptor and signaling modules with synthetic transmembrane domains that favor electrostatic interactions between the synthetic transmembrane domains in the cell membrane while eliminating or minimizing electrostatic interactions with the native transmembrane domains of immune receptors and signaling proteins. The modular chimeric receptors, which mimic the structure and signaling of native immune receptors, enable the distribution of the signaling domains across different cytoplasmic chains, display suitable surface expression as well as improved kinetics and sensitivities relative to current standard-of-care (SOC) CAR-based therapies.

### CROSS REFERENCE TO RELATED APPLICATIONS

The present application claims the benefit of U.S. provisional patent application No. 63/068,760 filed on Aug. 21, 2020, which is incorporated herein by reference in its entirety.

### TECHNICAL FIELD

The present invention generally relates to modular assembly receptors, such as modular chimeric antigen receptor (CAR) assemblies, and more particularly to the design of modular receptors for therapeutic applications such as CAR cell-based therapy, chronic inflammation and autoimmune diseases.

### BACKGROUND ART

The development of T-cell centric immunotherapeutic approaches, which make use of Chimeric Antigen Receptors (CARs), have shown remarkable efficacies in treating leukemia¹. CARs are type I transmembrane (TM) proteins comprised of an extra-cellular domain targeting tumor associated antigens, extracellular scaffold, a transmembrane domain akin to the scaffold, and a complex cytoplasmic domain recapitulating various signaling routes in T cells². Whereas first generation CARs included the cytoplasmic tail of the TCR-associated CD3zeta (CD3z or CD3ζ), later generation CARs employed additional signaling motifs fused to the CD3 domain such as to recapitulate co-stimulatory signaling routes from CD28, ICOS, or 4-1BB³⁻⁵. These stand of care CARs (SOC-CARs) show high efficacies in early response rate for treating relapsed B cell acute lymphoblastic leukemia (B-ALL) and Non-Hodgkin's Lymphoma¹.

However, although CAR-T therapies have proven to be quite successful as anti-tumor treatments, they also display undesired side effects. For instance, the CD19-directed CARs lead to severe B-cell aplasia⁶. This comes from the fact that current anti-CD19 CAR-T therapies, also target healthy cells as CD19 is expressed on normal B cells precursors. Two more severe complications linked with CAR-T therapy are neurotoxicity and cytokine release syndrome (CRS). While the molecular underpinnings of CAR-T associated neurotoxicity are still unclear, CRS can be linked to aberrant signaling of the CAR leading to an overwhelming activation of the immune system⁶. Possible cause for this can be linked to high-level surface expression as well as the use of sequential arrays of signaling motifs within a single cytoplasmic domain, which may cause steric hindrance between adaptor protein during receptor triggering and altering signaling cues. Indeed, phosphoproteomics studies have shown that both the CD28ζ and 4-1BBζ CARs display significant defects in signaling kinetics when compared to normal immune receptors, as well as aberrant hetero-dimerization for the CD28ζ CAR⁷⁻⁹. These signaling discrepancies between CARs and immune receptors are mostly likely due to the current architectures of these CARs. Whereas current CARs employ a linear array of various signaling cues, immune receptors such as the TCR, BCR as well as many NK cell activating receptors (NKR) are all found as modular receptors comprised of a ligand-binding (Rc) and signaling (Sig) modules¹⁰⁻¹⁶.

The transmembrane domains (TMDs) of CARs have received little attention the design of CARs. Most CARs incorporate the TMD sequence of the same protein from which the adjacent hinge or signalling domains were derived such as CD4, CD8α, CD28 or the TCR-associated ζ chain. However, These TMDs can engage in molecular interactions that drive self-association and/or assembly with the essential T cell proteins from which they were derived, which may impact CAR surface expression and functional properties.

There is thus a need for the development of novel CARs that better recapitulate normal immune receptor modular assembly and signaling kinetics, which could significantly improve treatment outcomes and mitigate current limitations and pitfalls observed in current single chain CAR technologies.

The present description refers to a number of documents, the content of which is herein incorporated by reference in their entirety.

### SUMMARY

The present disclosure provides the following items 1 to 92:
1. A modular chimeric receptor for expression in a target immune cell comprising:
a synthetic receptor module comprising an extracellular domain fused to a first synthetic transmembrane domain comprising a first positively charged amino acid;
a first synthetic signaling module comprising an intracellular signaling domain fused to a second synthetic transmembrane domain comprising a first negatively charged amino acid;
wherein the first positively charged amino acid and first negatively charged amino acid are positioned so that the electrostatic interactions between the first synthetic transmembrane domain and the second synthetic transmembrane domains in the target immune cell membrane are stronger than the electrostatic interactions with native transmembrane domains from immune receptors and/or native transmembrane domains from immune cell signaling proteins expressed by said target immune cell.
2. The modular chimeric receptor of item 1, wherein the first synthetic transmembrane domain is a variant of a native transmembrane domain from an immune receptor and/or wherein the second synthetic transmembrane domain is a variant of a native transmembrane domain from an immune cell signaling protein.
3. The modular chimeric receptor of item 2, wherein the first positively charged amino acid is located at a position that is 3 to 5 residues or 7 to 9 residues amino- or carboxy-terminal relative to the position of a positively charged amino acid in the native transmembrane domain from the immune receptor.
4. The modular chimeric receptor of item 3, wherein the first positively charged amino acid is located at a position that is 4 residues amino- or carboxy-terminal relative to the position of a positively charged amino acid in the native transmembrane domain from the immune receptor.
5. The modular chimeric receptor of any one of items 2 to 4, wherein the first negatively charged amino acid is located at a position that is 3 to 5 residues or 7 to 9 residues amino- or carboxy-terminal relative to the position of the negatively charged amino acid in the native transmembrane domain from the immune cell signaling protein.
6. The modular chimeric receptor of item 5, wherein the first negatively charged amino acid is located at a position that is 4 residues amino- or carboxy-terminal relative to the position of the negatively charged amino acid in the native transmembrane domain from the immune cell signaling protein.
7. The modular chimeric receptor of any one of items 1 to 6, wherein the first synthetic transmembrane domain comprises a threonine located at a position that is 4 residues amino- or carboxy-terminal relative to the position of the first positively charged amino acid.
8. The modular chimeric receptor of any one of items 1 to 7, wherein the second synthetic transmembrane domain comprises a threonine located at a position that is 4 residues amino- or carboxy-terminal relative to the position of the first negatively charged amino acid.
9. The modular chimeric receptor of any one of items 1 to 8, wherein the first synthetic transmembrane domain is a variant of the transmembrane domain (TM) of TRDC and comprises a sequence having at least 40% identity with the sequence VLGLRMLFAKTVAVNFLLTAKLFF (SEQ ID NO:1), wherein the K residue at position 10 and/or the K residue at position 21 is/are replaced by an uncharged amino acid, preferably a hydrophobic amino acid, and (i) at least one of: the A residue at position 13, the V residue at position 14, or the N residue at position 15 is replaced by a positively charged amino acid; or (ii) at least one of: the F residue at position 16, the L residue at position 17, or the L residue at position 18 is replaced by a positively charged amino acid.
10. The modular chimeric receptor of any one of items 1 to 8, wherein the first synthetic transmembrane domain is a variant of the TM of TRAC and comprises a sequence having at least 40% identity with the sequence VIGFRILLLKVAGFNLLMTLRLW (SEQ ID NO:2), wherein the R residue at position 5, the K residue at position 10 and/or the R residue at position 21 is/are replaced by an uncharged amino acid, preferably a hydrophobic amino acid, and (i) at least one of: the L residue at position 8 or the L residue at position 9 is replaced by a positively charged amino acid; (ii) at least one of: the I residue at position 6 or the L residue at position 7 is replaced by a positively charged amino acid; (iii) at least one of: the G residue at position 13, the F residue at position 14, or the N residue at position 15 is replaced by a positively charged amino acid; and/or (iv) at least one of: the L residue at position 16, the L residue at position 17, or the M residue at position 18 is replaced by a positively charged amino acid.
11. The modular chimeric receptor of any one of items 1 to 8, wherein the first synthetic transmembrane domain is a variant of the TM of TRBC1 or TRBC2 and comprises a sequence having at least 40% identity with the sequence ILLGKATLYAVLVSALVLMAMV (SEQ ID NO:3), wherein the K residue at position 5 is replaced by an uncharged amino acid, preferably a hydrophobic amino acid, and (i) at least one of: the L residue at position 8, the Y residue at position 9, or the A residue at position 10 is replaced by a positively charged amino acid; (ii) at least one of: the L residue at position 12, the V residue at position 13, or the S residue at position 14 is replaced by a positively charged amino acid; and/or (iii) at least one of: the L residue at position 16, the V residue at position 17, or the L residue at position 18 is replaced by a positively charged amino acid.
12. The modular chimeric receptor of any one of items 1 to 8, wherein the first synthetic transmembrane domain is a variant of the TM of TRGC1 and comprises a sequence having at least 40% identity with the sequence YYMYLLLLLKSVVYFAIITCCLL (SEQ ID NO:4), wherein the K residue at position 10 is replaced by an uncharged amino acid, preferably a hydrophobic amino acid, and (i) at least one of: the L residue at position 5, the L residue at position 6, or the L residue at position 7 is replaced by a positively charged amino acid; (ii) at least one of: the V residue at position 13, the Y residue at position 14, or the F residue at position 15 is replaced by a positively charged amino acid; and/or (iii) at least one of: the I residue at position 17, the I residue at position 18, or the T residue at position 19 is replaced by a positively charged amino acid.
13. The modular chimeric receptor of any one of items 1 to 8, wherein the first synthetic transmembrane domain is a variant of the TM of TRGC2 and comprises a sequence having at least 40% identity with the sequence YYTYLLLLLKSVVYFAIITCCLL (SEQ ID NO:5), wherein the K residue at position 10 is replaced by an uncharged amino acid, preferably a hydrophobic amino acid, and (i) at least one of: the L residue at position 5, the L residue at position 6, or the L residue at position 7 is replaced by a positively charged amino acid; (ii) at least one of: the V residue at position 13, the Y residue at position 14, or the F residue at position 15 is replaced by a positively charged amino acid; and/or (iii) at least one of: the I residue at position 17, the I residue at position 18, or the T residue at position 19 is replaced by a positively charged amino acid.
14. The modular chimeric receptor of any one of items 1 to 8, wherein the first synthetic transmembrane domain is a variant of the TM of NCTR1 and comprises a sequence having at least 40% identity with the sequence LLRMGLAFLVLVALVWFLV (SEQ ID NO:6), wherein the R residue at position 3 is replaced by an uncharged amino acid, preferably a hydrophobic amino acid, and (i) at least one of: the L residue at position 6, the A residue at position 7, or the F residue at position 8 is replaced by a positively charged amino acid; (ii) at least one of: the V residue at position 10, the L residue at position 11, or the V residue at position 12 is replaced by a positively charged amino acid; and/or (iii) at least one of: the V residue at position 14, the W residue at position 15, or the F residue at position 16 is replaced by a positively charged amino acid.
15. The modular chimeric receptor of any one of items 1 to 8, wherein the first synthetic transmembrane domain is a variant of the TM of NCTR2 and comprises a sequence having at least 40% identity with the sequence LVPVFCGLLVAKSLVLSALLV (SEQ ID NO:7), wherein the K residue at position 12 is replaced by an uncharged amino acid, preferably a hydrophobic amino acid, and (i) at least one of: the G residue at position 7, the L residue at position 8, or the L residue at position 9 is replaced by a positively charged amino acid; and/or (ii) at least one of: the V residue at position 11, the L residue at position 12, or the S residue at position 13 is replaced by a positively charged amino acid.
16. The modular chimeric receptor of any one of items 1 to 8, wherein the first synthetic transmembrane domain is a variant of the TM of NCTR3 and comprises a sequence having at least 40% identity with the sequence AGTVLLLRAGFYAVSFLSVAV (SEQ ID NO:8), wherein the R residue at position 8 is replaced by an uncharged amino acid, preferably a hydrophobic amino acid, and (i) at least one of: the T residue at position 3, the V residue at position 4, or the L residue at position 5 is replaced by a positively charged amino acid; (ii) at least one of: the F residue at position 11, the Y residue at position 12, or the A residue at position 13 is replaced by a positively charged amino acid; and/or (iii) at least one of: the S residue at position 15, the F residue at position 16, or the L residue at position 17 is replaced by a positively charged amino acid.
17. The modular chimeric receptor of any one of items 1 to 8, wherein the first synthetic transmembrane domain is a variant of the TM of KI2L4 and comprises a sequence having at least 40% identity with the sequence AVIRYSVAIILFTILPFFLLH (SEQ ID NO:9), wherein the R residue at position 4 is replaced by an uncharged amino acid, preferably a hydrophobic amino acid, and (i) at least one of: the V residue at position 7, the A residue at position 8, or the I residue at position 9 is replaced by a positively charged amino acid; (ii) at least one of: the L residue at position 11, the F residue at position 12, or the T residue at position 13 is replaced by a positively charged amino acid; and/or (iii) at least one of: the L residue at position 15, the P residue at position 16, or the F residue at position 18 is replaced by a positively charged amino acid.
18. The modular chimeric receptor of any one of items 1 to 8, wherein the first synthetic transmembrane domain is a variant of the TM of NKG2F and comprises a sequence having at least 40% identity with the sequence VLGIICIVLMATVLKTIVLIP (SEQ ID NO:10), wherein the K residue at position 15 is replaced by an uncharged amino acid, preferably a hydrophobic amino acid, and (i) at least one of: the M residue at position 10, the A residue at position 11, or the T residue at position 12 is replaced by a positively charged amino acid; and/or (ii) at least one of: the C residue at position 6, the I residue at position 7, or the V residue at position 8 is replaced by a positively charged amino acid.
19. The modular chimeric receptor of any one of items 1 to 8, wherein the first synthetic transmembrane domain is a variant of the TM of NKG2E and comprises a sequence having at least 40% identity with the sequence LTAEVLGIICIVLMATVLKTIVL (SEQ ID NO: 11), wherein the K residue at position 19 is replaced by an uncharged amino acid, preferably a hydrophobic amino acid, and (i) at least one of: the M residue at position 14, the A residue at position 15, or the T residue at position 16 is replaced by a positively charged amino acid; (ii) at least one of: the C residue at position 10, the I residue at position 11, or the V residue at position 12 is replaced by a positively charged amino acid; and/or (iii) at least one of: the L residue at position 6, the G residue at position 7, or the I residue at position 8 is replaced by a positively charged amino acid.
20. The modular chimeric receptor of any one of items 1 to 8, wherein the first synthetic transmembrane domain is a variant of the TM of NKG2D and comprises a sequence having at least 40% identity with the sequence PFFFCCFIAVAMGIRFIIMVA (SEQ ID NO:12), wherein the R residue at position 15 is replaced by an uncharged amino acid, preferably a hydrophobic amino acid, and (i) at least one of: the V residue at position 10, the A residue at position 11, or the M residue at position 12 is replaced by a positively charged amino acid; and/or (ii) at least one of: the C residue at position 6, the F residue at position 7, or the I residue at position 8 is replaced by a positively charged amino acid.
21. The modular chimeric receptor of any one of items 1 to 8, wherein the first synthetic transmembrane domain is a variant of the TM of NKG2C and comprises a sequence having at least 40% identity with the sequence VLGIICIVLMATVLKTIVLIPFL (SEQ ID NO:13), wherein the K residue at position 15 is replaced by an uncharged amino acid, preferably a hydrophobic amino acid, and (i) at least one of: the M residue at position 10, the A residue at position 11, or the T residue at position 12 is replaced by a positively charged amino acid; and/or (ii) at least one of: the C residue at position 6, the I residue at position 7, or the V residue at position 8 is replaced by a positively charged amino acid.
22. The modular chimeric receptor of any one of items 1 to 8, wherein the first synthetic transmembrane domain is a variant of the TM of KI2S1 and comprises a sequence having at least 50% identity with the sequence VLIGTSVVKIPFTILLFFL (SEQ ID NO:14), wherein the K residue at position 9 is replaced by an uncharged amino acid, preferably a hydrophobic amino acid, and (i) at least one of: the G residue at position 4, the T residue at position 5, or the S residue at position 6 is replaced by a positively charged amino acid; and/or (ii) at least one of: the F residue at position 12, the T residue at position 13, or the I residue at position 14 is replaced by a positively charged amino acid.
23. The modular chimeric receptor of any one of items 1 to 8, wherein the first synthetic transmembrane domain is a variant of the TM of KI2S2 or KI2S4 and comprises a sequence having at least 40% identity with the sequence VLIGTSVVKIPFTILLFFLL (SEQ ID NO:15), wherein the K residue at position 9 is replaced by an uncharged amino acid, preferably a hydrophobic amino acid, and (i) at least one of: the G residue at position 4, the T residue at position 5, or the S residue at position 6 is replaced by a positively charged amino acid; and/or (ii) at least one of: the F residue at position 12, the T residue at position 13, or the I residue at position 14 is replaced by a positively charged amino acid.
24. The modular chimeric receptor of any one of items 1 to 8, wherein the first synthetic transmembrane domain is a variant of the TM of KI2S3 or KI2S5 and comprises a sequence having at least 40% identity with the sequence VLIGTSVVKLPFTILLFFL (SEQ ID NO:16), wherein the K residue at position 9 is replaced by an uncharged amino acid, preferably a hydrophobic amino acid, and (i) at least one of: the G residue at position 4, the T residue at position 5, or the S residue at position 6 is replaced by a positively charged amino acid; and/or (ii) at least one of: the F residue at position 12, the T residue at position 13, or the I residue at position 14 is replaced by a positively charged amino acid.
25. The modular chimeric receptor of item 24, wherein the first synthetic transmembrane domain comprises the amino acid sequence VLIGTSVVLLPFKILLFFLL (SEQ ID NO:32), VLIILLVGTSVVKLLLFFLL (SEQ ID NO:33), VLIGTSVVTLPFKILLFFLL (SEQ ID NO:34), VLILLLLLLLLLKLLLFFLL (SEQ ID NO:35), VLILLLLGLLLLKLLLFFLL (SEQ ID NO:36), VLILLLLLALLLKLLLFFLL (SEQ ID NO:37) or VLILLLLLTLLLKLLLFFLL (SEQ ID NO:38), preferably SEQ ID NO:38.
26. The modular chimeric receptor of any one of items 1 to 8, wherein the first synthetic transmembrane domain is a variant of the TM of K13S1 and comprises a sequence having at least 40% identity with the sequence ILIGTSVVKIPFTILLFFLL (SEQ ID NO:17), wherein the K residue at position 9 is replaced by an uncharged amino acid, preferably a hydrophobic amino acid, and (i) at least one of: the G residue at position 4, the T residue at position 5, or the S residue at position 6 is replaced by a positively charged amino acid; and/or (ii) at least one of: the F residue at position 12, the T residue at position 13, or the I residue at position 14 is replaced by a positively charged amino acid.
27. The modular chimeric receptor of any one of items 1 to 8, wherein the first synthetic transmembrane domain is a variant of the TM of TREM1 and comprises a sequence having at least 40% identity with the sequence IVILLAGGFLSKSLVFSVLFA (SEQ ID NO:18), wherein the K residue at position 12 is replaced by an uncharged amino acid, preferably a hydrophobic amino acid, and (i) at least one of: the G residue at position 7, the G residue at position 8, or the F residue at position 9 is replaced by a positively charged amino acid; and/or (ii) at least one of: the V residue at position 15, the F residue at position 16, or the S residue at position 17 is replaced by a positively charged amino acid.
28. The modular chimeric receptor of any one of items 1 to 8, wherein the first synthetic transmembrane domain is a variant of the TM of TREM2 and comprises a sequence having at least 40% identity with the sequence ILLLLACIFLIKILAASALWA (SEQ ID NO:19), wherein the K residue at position 12 is replaced by an uncharged amino acid, preferably a hydrophobic amino acid, and (i) at least one of: the C residue at position 7, the I residue at position 8, or the F residue at position 9 is replaced by a positively charged amino acid; and/or (ii) at least one of: the A residue at position 15, the A residue at position 16, or the S residue at position 17 is replaced by a positively charged amino acid.
29. The modular chimeric receptor of any one of items 1 to 8, wherein the first synthetic transmembrane domain is a variant of the TM of GPVI and comprises a sequence having at least 40% identity with the sequence GNLVRICLGAVILIILAGFLA (SEQ ID NO:20), wherein the R residue at position 5 is replaced by an uncharged amino acid, preferably a hydrophobic amino acid, and (i) at least one of: the L residue at position 8, the G residue at position 9, or the A residue at position 10 is replaced by a positively charged amino acid; and/or (ii) at least one of: the I residue at position 12, the L residue at position 13, or the I residue at position 14 is replaced by a positively charged amino acid; and/or (iii) at least one of: the L residue at position 16, the A residue at position 17, or the G residue at position 18 is replaced by a positively charged amino acid.
30. The modular chimeric receptor of any one of items 1 to 29, wherein the second synthetic transmembrane domain is a variant of the TM of CD3D and comprises a sequence having at least 40% identity with the sequence GIIVTDVIATLLLALGVFCFA (SEQ ID NO:21), wherein the D residue at position 6 is replaced by an uncharged amino acid, preferably a hydrophobic amino acid, and (i) at least one of: the A residue at position 9, the T residue at position 10, or the L residue at position 11 is replaced by a negatively charged amino acid; and/or (ii) at least one of: the L residue at position 13, the A residue at position 14, or the L residue at position 15 is replaced by a negatively charged amino acid.
31. The modular chimeric receptor of any one of items 1 to 29, wherein the second synthetic transmembrane domain is a variant of the TM of CD3E and comprises a sequence having at least 40% identity with the sequence VSVATIVIVDICITGGLLLLVYYWS (SEQ ID NO:22), wherein the D residue at position 10 is replaced by an uncharged amino acid, preferably a hydrophobic amino acid, and (i) at least one of: the T residue at position 5, the I residue at position 6, or the V residue at position 7 is replaced by a negatively charged amino acid; (ii) at least one of: the I residue at position 13, the T residue at position 14, or the G residue at position 15 is replaced by a negatively charged amino acid; and/or (iii) at least one of: the L residue at position 17, the L residue at position 18, or the L residue at position 19 is replaced by a negatively charged amino acid.
32. The modular chimeric receptor of any one of items 1 to 29, wherein the second synthetic transmembrane domain is a variant of the TM of CD3G and comprises a sequence having at least 40% identity with the sequence GFLFAEIVSIFVLAVGVYFIA (SEQ ID NO:23), wherein the E residue at position 6 is replaced by an uncharged amino acid, preferably a hydrophobic amino acid, and (i) at least one of: the S residue at position 9, the I residue at position 10, or the F residue at position 11 is replaced by a negatively charged amino acid; and/or (ii) at least one of: the L residue at position 13, the A residue at position 14, or the V residue at position 15 is replaced by a negatively charged amino acid.
33. The modular chimeric receptor of any one of items 1 to 29, wherein the second synthetic transmembrane domain is a variant of the TM of CD3Z and comprises a sequence having at least 40% identity with the sequence LCYLLDGILFIYGVILTALFL (SEQ ID NO:24), wherein the D residue at position 6 is replaced by an uncharged amino acid, preferably a hydrophobic amino acid, and (i) at least one of: the L residue at position 9, the F residue at position 10, or the I residue at position 11 is replaced by a negatively charged amino acid; and/or (ii) at least one of: the G residue at position 13, the V residue at position 14, or the I residue at position 15 is replaced by a negatively charged amino acid.
34. The modular chimeric receptor of any one of items 1 to 29, wherein the second synthetic transmembrane domain is a variant of the TM of HCST/DAP10 and comprises a sequence having at least 40% identity with the sequence LLAGLVAADAVASLLIVGAVF (SEQ ID NO:25), wherein the D residue at position 9 is replaced by an uncharged amino acid, preferably a hydrophobic amino acid, and (i) at least one of: the G residue at position 4, the L residue at position 5, or the V residue at position 6 is replaced by a negatively charged amino acid; (ii) at least one of: the A residue at position 12, the S residue at position 13, or the L residue at position 14 is replaced by a negatively charged amino acid; and/or (iii) at least one of: the I residue at position 16, the V residue at position 17, or the G residue at position 18 is replaced by a negatively charged amino acid.
35. The modular chimeric receptor of any one of items 1 to 29, wherein the second synthetic transmembrane domain is a variant of the TM of TYROBP/DAP12 and comprises a sequence having at least 40% identity with the sequence VLAGIVMGDLVLTVLIALAVYFL (SEQ ID NO:26), wherein the D residue at position 9 is replaced by an uncharged amino acid, preferably a hydrophobic amino acid, and (i) at least one of: the G residue at position 4, the I residue at position 5, or the V residue at position 6 is replaced by a negatively charged amino acid; (ii) at least one of: the L residue at position 12, the T residue at position 13, or the V residue at position 14 is replaced by a negatively charged amino acid; and/or (iii) at least one of: the I residue at position 16, the A residue at position 17, or the L residue at position 18 is replaced by a negatively charged amino acid.
36. The modular chimeric receptor of item 35, wherein the second synthetic transmembrane domain comprises the sequence VLAGIVMGALVLDVLITLAVYFL (SEQ ID NO:39), VLALAVLGIVMGDVLITLAVYFL (SEQ ID NO:40) or VLAGDVMGTLVLIVLIALAVYFL (SEQ ID NO:41).
37. The modular chimeric receptor of any one of items 1 to 29, wherein the second synthetic transmembrane domain is a variant of the TM of CD79A and comprises a sequence having at least 40% identity with the sequence IITAEGIILLFCAVVPGTLLLF (SEQ ID NO:27), wherein the E residue at position 5 is replaced by an uncharged amino acid, preferably a hydrophobic amino acid, and (i) at least one of: the I residue at position 8, the L residue at position 9, or the L residue at position 10 is replaced by a negatively charged amino acid; (ii) at least one of: the C residue at position 12, the A residue at position 13, or the V residue at position 14 is replaced by a negatively charged amino acid; and/or (iii) at least one of: the G residue at position 16, the T residue at position 17, or the L residue at position 18 is replaced by a negatively charged amino acid.
38. The modular chimeric receptor of any one of items 1 to 29, wherein the second synthetic transmembrane domain is a variant of the TM of FCERG and comprises a sequence having at least 40% identity with the sequence LCYILDAILFLYGIVLTLLYC (SEQ ID NO:28), wherein the D residue at position 6 is replaced by an uncharged amino acid, preferably a hydrophobic amino acid, and (i) at least one of: the L residue at position 9, the F residue at position 10, or the L residue at position 11 is replaced by a negatively charged amino acid; (ii) at least one of: the G residue at position 13, the I residue at position 14, or the V residue at position 15 is replaced by a negatively charged amino acid; and/or (iii) at least one of: the T residue at position 17, the L residue at position 18, or the L residue at position 19 is replaced by a negatively charged amino acid.
39. The modular chimeric receptor of any one of items 1 to 29, wherein the second synthetic transmembrane domain is a variant of the TM of FCERA and comprises a sequence having at least 40% identity with the sequence FFIPLLVVILFAVDTGLFI (SEQ ID NO:29), wherein the D residue at position 14 is replaced by an uncharged amino acid, preferably a hydrophobic amino acid, and (i) at least one of: the I residue at position 9, the L residue at position 10, or the F residue at position 11 is replaced by a negatively charged amino acid; and/or (ii) at least one of: the L residue at position 5, the L residue at position 6, or the V residue at position 7 is replaced by a negatively charged amino acid.
40. The modular chimeric receptor of any one of items 1 to 29, wherein the second synthetic transmembrane domain is a variant of the TM of FCG3A and comprises a sequence having at least 40% identity with the sequence VSFCLVMVLLFAVDTGLYFSV (SEQ ID NO:30), wherein the D residue at position 14 is replaced by an uncharged amino acid, preferably a hydrophobic amino acid, and (i) at least one of: the L residue at position 9, the L residue at position 10, or the F residue at position 11 is replaced by a negatively charged amino acid; (ii) at least one of: the L residue at position 5, the V residue at position 6, or the M residue at position 7 is replaced by a negatively charged amino acid; and/or (iii) at least one of: the L residue at position 17, the Y residue at position 18, or the F residue at position 19 is replaced by a negatively charged amino acid.
41. The modular chimeric receptor of any one of items 1 to 29, wherein the second synthetic transmembrane domain is a variant of the TM of FCRL1 and comprises a sequence having at least 50% identity with the sequence GVIEGLLSTLGPATVALLFCY (SEQ ID NO:31), wherein the E residue at position 4 is replaced by an uncharged amino acid, preferably a hydrophobic amino acid, and (i) at least one of: the L residue at position 7, the S residue at position 8, or the T residue at position 9 is replaced by a negatively charged amino acid; (ii) at least one of: the G residue at position 11, the P residue at position 12, or the A residue at position 13 is replaced by a negatively charged amino acid; and/or (iii) at least one of: the V residue at position 15, the A residue at position 16, or the L residue at position 17 is replaced by a negatively charged amino acid.
42. The modular chimeric receptor of any one of items 1 to 41, wherein the first synthetic transmembrane domain is a variant of the TM of KI2S3 and the second synthetic transmembrane domain is a variant of the TM of DAP12 or CD3Z.
43. The modular chimeric receptor of any one of items 1 to 41, wherein the first synthetic transmembrane domain is a variant of the TM of NKG2C and the second synthetic transmembrane domain is a variant of the TM of DAP12 or CD3Z.
44. The modular chimeric receptor of any one of items 1 to 43, wherein the first synthetic transmembrane domain comprises two positively-charged residues and the modular chimeric receptor comprises a second signaling module comprising a third synthetic transmembrane domain, wherein the third synthetic transmembrane domain comprises a second negatively charged amino acid and is a variant of a native transmembrane domain from an immune cell signaling protein, and wherein the second negatively charged amino acid is positioned so that the electrostatic interactions between the third synthetic transmembrane domain and the first synthetic transmembrane domain in the target immune cell membrane are stronger than the electrostatic interactions between the third synthetic transmembrane domain and the native transmembrane domain from the immune receptor.
45. The modular chimeric receptor of any one of items 1 to 44, wherein the extracellular domain of the synthetic receptor module comprises one or more ligand- or antigen-binding domains.
46. The modular chimeric receptor of item 45, wherein the extracellular domain of the synthetic receptor module comprises two ligand- or antigen-binding domains.
47. The modular chimeric receptor of item 45 or 46, wherein the extracellular domain of the synthetic receptor module comprises an antigen-binding portion of an antibody molecule.
48. The modular chimeric receptor of item 47, wherein the extracellular domain of the synthetic receptor module comprises a single-chain antibody fragment (scFv) or a single-domain antibody (sdAb).
49. The modular chimeric receptor of item 48, wherein the extracellular domain of the synthetic receptor module comprises two scFvs or sdAbs.
50. The modular chimeric receptor of any one of items 45 to 49, wherein the antigen is a protein expressed at the surface of tumor cells.
51. The modular chimeric receptor of item 50, wherein the protein expressed at the surface of tumor cells is CD19.
52. The modular chimeric receptor of item 51, wherein the extracellular domain of the synthetic receptor module comprises the amino acid sequence of SEQ ID NO:65.
53. The modular chimeric receptor of any one of items 1 to 52, wherein the synthetic receptor module further comprises a polypeptide linker or spacer between the extracellular domain and the first transmembrane domain.
54. The modular chimeric receptor of item 53, wherein the polypeptide linker or spacer comprises a portion of the extracellular domain of human CD8alpha.
55. The modular chimeric receptor of item 54, wherein the polypeptide linker or spacer comprises the amino acid sequence of SEQ ID NO:66.
56. The modular chimeric receptor of any one of items 1 to 55, wherein the synthetic receptor module further comprises an intracellular domain.
57. The modular chimeric receptor of item 56, wherein the intracellular domain of the synthetic receptor module comprises a sequence of an intracellular domain of a costimulatory immune receptor.
58. The modular chimeric receptor of item 57, wherein the costimulatory immune receptor is CD28, 4-1BB, OX40, or ICOS, preferably CD28.
59. The modular chimeric receptor of item 58, wherein the intracellular domain of the synthetic receptor module comprises the amino acid sequence of SEQ ID NO:68.
60. The modular chimeric receptor of any one of items 1 to 59, wherein the intracellular signaling domain of the first synthetic signaling module comprises a sequence of an intracellular domain of an immune cell signaling protein and/or of a costimulatory immune receptor.
61. The modular chimeric receptor of item 60, wherein the intracellular signaling domain of the first synthetic signaling module comprises a sequence of the intracellular domain of DAP12 or CD3Z.
62. The modular chimeric receptor of item 61, wherein the intracellular signaling domain of the first synthetic signaling module comprises the amino acid sequence of SEQ ID NO:68, 69, 70 or 71.
63. A nucleic acid or a plurality of nucleic acids comprising nucleotide sequences encoding the receptor module and signaling module(s) defined in any one of items 1 to 62.
64. The nucleic acid or plurality of nucleic acids of item 63, wherein said nucleic acid or plurality of nucleic acids is/are present in one or more plasmids or vectors.
65. The nucleic acid or plurality of nucleic acids of item 64, wherein said one or more plasmids or vectors is/are viral vectors such as lentiviral vectors.
66. An immune cell expressing the modular chimeric receptor of any one of items 1 to 65.
67. The immune cell of item 66, wherein the immune cell is a T cell, such as a CD8+ T cell, or a natural killer (NK) cell.
68. A composition comprising the immune cell of item 66 or 67 and at least one pharmaceutically acceptable carrier or excipient.
69. A method for treating a disease, condition or disorder in a subject, the method comprising administering the immune cell of item 66 or 67 or the composition of item 68 to the subject.
70. The method of item 69, wherein the disease, condition or disorder is cancer, an autoimmune or inflammatory disease, or an infectious disease.
71. The method of item 70, wherein the disease, condition or disorder is a hematologic cancer, such as a B cell-derived cancer.
72. The method of item 70 or 71, wherein the cancer is B-cell lymphoma (BCL), mantle cell lymphoma (MCL), multiple myeloma (MM), or acute lymphoblastic leukemia (ALL).
73. The method of any one of items 70 to 72, wherein the cancer is a CD19-expressing cancer.
74. A method for inducing the suppression of a target cell, the method comprising contacting the target cell with the immune cell of item 66 or 67, or with a composition comprising the immune cell, wherein the target cell expresses at its surface an antigen recognizes by the extracellular domain of the receptor module of the modular chimeric receptor.
75. The method of item 74, wherein the target cell is a tumor cell.
76. The method of item 75, wherein the tumor cell is a B cell.
77. The immune cell of item 66 or 67 or the composition of item 68, for use in treating a disease, condition or disorder in a subject.
78. The immune cell or composition for use according to item 77, wherein the disease, condition or disorder is cancer, an autoimmune or inflammatory disease, or an infectious disease.
79. The immune cell or composition for use according to item 78, wherein the disease, condition or disorder is a hematologic cancer, such as a B cell-derived cancer.
80. The immune cell or composition for use according to item 78 or 79, wherein the cancer is B-cell lymphoma (BCL), mantle cell lymphoma (MCL), multiple myeloma (MM), or acute lymphoblastic leukemia (ALL).
81. The immune cell or composition for use according to any one of items 78 to 80, wherein the cancer is a CD19-expressing cancer.
82. The immune cell of item 66 or 67 or a composition comprising the immune cell, for use in the suppression of a target cell expressing at its surface an antigen recognizes by the extracellular domain of the receptor module of the modular chimeric receptor.
83. The immune cell or composition for use according to item 82, wherein the target cell is a tumor cell.
84. The immune cell or composition for use according to item 83, wherein the tumor cell is a B cell.
85. Use of the immune cell of item 66 or 67 or the composition of item 68, for the manufacture of a medicament for treating a disease, condition or disorder in a subject.
86. The use according to item 85, wherein the disease, condition or disorder is cancer, an autoimmune or inflammatory disease, or an infectious disease.
87. The use according to item 85, wherein the disease, condition or disorder is a hematologic cancer, such as a B cell-derived cancer.
88. The use according to item 86 or 87, wherein the cancer is B-cell lymphoma (BCL), mantle cell lymphoma (MCL), multiple myeloma (MM), or acute lymphoblastic leukemia (ALL).
89. The use according to any one of items 86 to 88, wherein the cancer is a CD19-expressing cancer.
90. Use of the immune cell of item 66 or 67 or a composition comprising the immune cell for the manufacture of a medicament for suppressing a target cell expressing at its surface an antigen recognizes by the extracellular domain of the receptor module of the modular chimeric receptor.
91. The use according to item 90, wherein the target cell is a tumor cell.
92. The use according to item 91, wherein the tumor cell is a B cell.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIGS. 1A-D show the design of novel transmembrane binding registry (TBR) to insure exclusive assembly of modular chimeric antigen receptor components. FIG. 1A: Schematic representation of the bicistronic cassette design for the receptor TBR screen also containing DAP12-targeting shRNA cassette. U6 promotor drives the expression of DAP12-targeting shRNA. The elF1a promotor drives the expression of a 2A peptide-based bi-cistronic cassette containing the extracellular (EC) and cytoplasmic (CD) portions of the receptor module and signalling module. Unique restriction sites were introduced between the EC and CD to enable the rapid insertions on the various transmembrane domains (TM) to be tested using homologous recombination. An N-terminal tag was added to the receptor module in order to facilitate FACS analyses, and the portion of DAP12 targeted by the shRNA was codon-swapped (*) as to not be targeted by the shRNA. FIG. 1B: The charged amino acids responsible for directing ternary TM assembly were moved four (4) positions up or down within the TM helix in order to change the binding registry. The helper threonine (T) in the receptor module helix was maintained at equidistance from the negatively charged Aspartate (D). The positively-charge Lysine (K) within the receptor modules' TM helix was moved similarly. The poly-leucine (pL+4) was created by replacing 9 core amino acids with leucine residues to create a synthetic sequence. EC=extra-cellular domain, CD=cytoplasmic domain. FIGS. 1C-D: Schematic representation of the modular CAR architecture. The ligand-binding modules (Rc) and the signaling module (Sig) specifically assemble to TM specific electrostatic interactions (D) that is herein referred to as the TM binding registry (TBR). Receptor assembly should occur only when the position of the charged amino acids within the TM of the Rc and Sig are matched to allow electrostatic interactions. Black circles represent negative charges (Glu) and white circles represent positive charges (Lys).

FIGS. 2A-E are graphs showing that the receptor module transmembrane electrostatic charge shift leads to preferential assembly to electrostatic charge shifted signalling module in the (+4) position. FIG. 2A: NKG2C/DAP12 TM binding registry screen was performed by transducing SH1-J cells to express the indicated constructs. 72 hours following transduction, cells were stained for NKG2C surface expression and analyzed by FACS. Numbers within each FACS plot indicate the relative expression level to that of the WT/WT condition. Mock transduction in dark grey, Screen transduction in light grey. Results representative of 3 independent transduction experiments. FIG. 2B: Jurkat WT cells were dually transduced with the KIR2DS3 +4 and DAP12 +4 constructs (see FIGS. 1B and C) and labeled with tetramerized CD19-Alexa647® to assess surface expression. Single positive and double positive populations based on mCherry and ZsGreen were analyzed for surface expression of the CD19-specific modular CARs. FIG. 2C: Surface expression of the CARs derived from the screen was quantified and compared to the surface expression of the CD19-KIRDS/DAP12 receptor (RS-WT/DS-WT). Data presented as relative expression to the CD19-KIRSA/DAP12. Quantification of Rc modules only population (DS-None) was also done to ascertain potential leakiness of the novel RS modules. Lack of assembly between endogenous KIR module (RS-WT) with the +4 and −4 synthetic DS modules is evident. FIG. 2D: To further highlight the importance of the position of the charged residue, a secondary screen was performed where all but the positive charged amino acids in the RS binding registry were mutated to leucines (RS modules 3, 4 and 5). Receptor assembly and surface expression was determined as before. FIG. 2E: Further RS sequence optimization was performed to identify stabilizing amino acids derived from the most promising candidates RS1 and RS4 as well as a modified DS modules module to accommodate these changes. Receptor assembly and surface expression was determined as before. Sequence information can be found in Table I.

FIGS. 3A-F show that the signaling of representative modular CARs of the present disclosure is robust, stable, and mimics normal immune receptor signaling. FIG. 3A is a schematic representation of the various modular CAR architectures and signaling domains used. FIG. 3B shows surface expression of the various modular CARs tested using fluorescently-labeled tetramerized CD19. The RS1- and RS4-derived improved Rc modules, RS1.3 and RS4.6, respectively, were used in these assays. FIG. 3C: Activating potential of the various modular CARs tested using CD19-expressing B cell leukemia in a co-culture assay. Data presented as relative surface expression of the modular CARs relative to the KIRD2DS3/DAP12 receptor (WT/WT-DAP). Activated status of the modular CAR-expressing Jurkat cells was monitored looking for the surface expression of the activation marker CD69 using fluorescently labeled anti-CD69 and performing FACS analysis. FIG. 3D: Data of experiments illustrated in C presented as relative fraction of cells expressing the activation marker CD69 at the surface of cells 16 hours following co-culture with CD19-positive B cell leukemic cells. FIG. 3E: Characterization of the effects of CAR expression on cell phenotype, viability and sustainability (N=3). 72 hours after transduction (day0), cells were monitored for CAR and basal CD69 surface expression over the course of 9 days to determine the level of basal receptor activity. Cells were also stimulated repeatedly starting day 3 to test for the sustained activation ability of the CARs and cell viability. In these assays, the CD19-specific KIRDS/DAP12, RS4.6/DS2-Z, and SOC-CAR CD28Z were assayed. FIG. 3F: Comparative assessment of the signaling capabilities of the modular CAR RS4.6/DS2-Z and TCR following engagement with cognate target cells. For RS4.6/DS2-Z triggering, CD19 coated beads were used, whereas for TCR triggering, while anti-CD3/CD28 triggering was used to follow TCR-based signaling. Results presented as ratios between loading control and phospho-specific signals, normalized to unstimulated conditions. Phospho signals from CD3zeta, ZAP70 and LAT were detected using phospho-specific antibodies.

FIGS. 4A-E show that representative modular CARs of the present disclosure outperform standard-of-care CARs in vitro. FIG. 4A is a schematic representation of the various CARs used in these assays based on their scaffolds (Scaff.) The signaling domains present in the various cytoplasmic domains are also illustrated and highlights the added CD28 signaling moiety to the Rc module. FIG. 4B: Surface CAR expression was determined using fluorescently-labeled tetramerized CD19. Average geometric mean fluorescence signal of CD19-labeling (GeoMFI) for assembled and non-assembled (Rc module alone) modular CAR as well as 1^{st} and 2^{nd} generation CARs are presented (N=3). FIG. 4C: Stability of surface expression RS4.6/DSZ and RS28/DSZ was measure starting 72 hours post-transduction (day 0) and assayed for 9 days using labeled CD19 as done previously. Sustained activation potential of the RS28/DSZ CAR was assayed starting at day 3 until day 9 as done previously and monitored for CD69 expression. FIG. 4D: CD19 surface expression was determined for three separate NHL lymphoma cell lines Toledo (TOL), RL, and HT used for the cytotoxic assay using fluorescently-labeled anti-CD19 antibody. FIG. 4E: Assessment of the cytolytic abilities of primary human CD8 T cells expressing the CARs indicated in the assays and target the indicated cell lines. Previously, the lymphoma cell lines were transduced to express fluorescent protein Ametrine (405 nm Ex, 530 nm Em) in the cytoplasm. Cytolytic assay is performed by co-incubating activated CAR-expressing CD8 T cells with target cells at a 5:1 ratio for 32-36 hours. Following incubation, plates are spun and the supernatant is recovered for the presence of soluble Ametrine using a plate reader compatible spectrofluorometer. Cell lysis efficiency is determined by comparing Ametrine levels in the supernatant of cytolytic assay to that of detergent-treated target cells. As control, cells expressing only the RS28 module were also tested (Rc). P values (<0,001) were obtained using Two-Way ANOVA and those presented in the graph indicate the statistical significance between the modular CARs and the 28Z per cell line.

FIGS. 5A-I show that modular CAR assembly and functions are not restricted to KIR2DS3 scaffold. FIG. 5A is a schematic representation of the various CARs used to demonstrate this. The various Receptor Modules (Rc.Mod.) are indicated as well as the extracellular (EC) scaffolds, transmembrane registry combinations (TBR) and cytoplasmic signaling domains (Cyto.D) used for experiments depicted in FIGS. 5B-C. As comparison to the KIR2DS3 EC scaffolds, the SOC-CAR CD8alpha (CD8α) was used as EC scaffold in the new modular CARs. FIG. 5B: Surface expression of the various CARs tested containing different scaffolds within the Rc.Mods and TBR combinations with Sig.Mod. Data presented as relative to the RS28/DS pair. FIG. 5C: Activating potential of the various modular CARs tested using CD19-expressing B cell leukemia in a co-culture assay. Activated status of the modular CAR-expressing Jurkat cells was monitored looking for the surface expression of the activation marker CD69 using fluorescently labeled anti-CD69 and performing FACS analysis. Data presented as relative fraction of cells expressing the activation marker CD69 at the surface of cells 16 hours following co-culture with CD19-positive B cell leukemic cells. FIG. 5D: Schematic representation of the expression cassette driving the expression of the D8-28/DS modular CAR under the control of a single promoter producing a polycistronic mRNA, which yields two separate proteins following P2A peptide cleavage. FIG. 5E: Assessment of the signaling capabilities of the D8-28/DS modular CAR following activation with CD19-positive B-ALL leukemic cells. Phospho signals from CD3zeta, ZAP70 and LAT were detected using phospho-specific antibodies. Loading controls for ZAP70 and LAT are also presented. FIG. 5F: Quantification (N=3) of signaling kinetics of the D8-28/DS modular CAR following its activation by encountering CD19-positive B-ALL leukemic cells in vitro. FIG. 5G: Assessment of basal and activation induced surface expression of the activation marker CD69 at day 6 post-transduction. Cells were activated by co-culture with CD19-positive B-ALL leukemic cells for 16 hours, then stained with fluorescently labeled anti-CD69, and then analyzed by FACS. FIG. 5H Analysis (N=3) of the surface expression of CD69 in non-activated (Resting) or following 16 hours of co-culture with CD129-positive B-ALL leukemic cells (+B-ALL). FIG. 5I: Comparative assessment of the surface expression of the D8-28/DS modular CAR and the SOC-CAR using fluorescently-labeled tetramerized CD19. Data presented as Geometric Mean Fluorescence (GMFI).

FIGS. 6A-C show that modular CARs enable the formation of stable immune synapses and promote rapid tumor cell killing in vitro. Primary human T cells were transduced with the D8-28/DS construct illustrated in FIG. 5A and sorted based on fluorescence as well as CD4 or CD8 expression. FIG. 6A-B: CD4 T cells expressing the D8-28/DS modular CAR that express cytoplasmic GFP (#) were co-incubated with Lact-C2-RFP expressing CD19-positive B-ALL leukemic cell (*) and imaged up to 45 mins using confocal microscopy. FIG. 6A: Representative example of early synapse formation between CD4 T cells expressing the modular CAR and B-ALL leukemic cell. FIG. 6B: Long-term imaging of immune synapse between CD4 T cells expressing the modular CAR and B-ALL leukemic cell from initiation, to stable formation and resolve. FIG. 6C: Representative example of immune synapse formation and tumor cell killing of B-ALL leukemic cell by CD8 T cells expressing the modular CD19-specific CAR D8-28/DS. Tumor cell killing can be observed by rapid collapse of the target cell shape and size as well as an increase in vesicular staining of the Lact-C2-RFP reporter. All time stamps are in minutes.

### DETAILED DISCLOSURE

Unless otherwise defined herein, scientific and technical terms used in connection with this disclosure shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the disclosure are generally performed according to conventional methods well-known in the art and as described in various general and more specific references that are cited and discussed throughout the specification unless otherwise indicated. See, e.g.: Sambrook J. & Russell D. Molecular Cloning: A Laboratory Manual, 3^{rd} ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual; Cold Spring Harbor Laboratory Press, Cold Spring Harbor, N.Y. (1998); and Coligan et al., Short Protocols in Protein Science, Wiley, John & Sons, Inc. (2003). Any enzymatic reactions or purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

The use of the terms “a” and “an” and “the” and similar referents in the context of describing the technology (especially in the context of the claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

The use of any and all examples, or exemplary language (“e.g.”, “such as”) provided herein, is intended merely to better illustrate the technology and does not pose a limitation on the scope of the claimed invention unless otherwise claimed.

No language in the specification should be construed as indicating any non-claimed element as essential to the practice of the claimed invention.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

In the studies described herein, the present inventor has developed modular chimeric receptors comprising ligand-binding and signaling modules comprising compatible synthetic transmembrane domains. The synthetic transmembrane domains were designed by shifting the position of the positively and negatively charged residues involved in the electrostatic interaction between ligand-binding and signaling modules within the membrane, which allows the ligand-binding and signaling modules to assemble together to form a functional chimeric receptor, without interfering or competing with endogenous immune receptors.

Accordingly, in a first aspect, the present disclosure provides a modular chimeric receptor for expression in a target immune cell comprising:
a synthetic receptor module comprising an extracellular domain (e.g., an antigen-binding domain) fused to a first synthetic transmembrane domain comprising a first positively charged amino acid;
a first synthetic signaling module comprising an intracellular domain (e.g., an immune cell signaling domain) fused to a second synthetic transmembrane domain comprising a first negatively charged amino acid;
wherein the first positively charged amino acid and first negatively charged amino acid are positioned so that the electrostatic interactions between the first synthetic transmembrane domain and the second synthetic transmembrane domains in the target immune cell membrane are stronger than the electrostatic interactions with the native transmembrane domain from the immune receptor and/or native transmembrane domain from the immune cell signaling protein.

Stronger electrostatic interactions mean that the ability of the first synthetic TM and the second synthetic TM to bind to each other (i.e. to assemble) in the target immune cell membrane is better than their ability to bind to the native TM from the immune receptor and/or native TM from the immune cell signaling protein. Since modular immune receptors require assembly in the cell membrane for expression at the cell surface, the ability of the modules (synthetic or native) to bind to each other (i.e. to assemble) in the cell membrane may be assessed by measuring the level of expression of the modular receptors at the cell surface (as shown in the Examples below), with a higher surface expression of the entirely synthetic modules (i.e. comprising the first and second synthetic transmembrane domains) relative to modules comprising a native TM indicating stronger electrostatic interactions between the first and second synthetic transmembrane domains. It is to be understood that the electrostatic interactions between the first synthetic transmembrane domain and the second synthetic transmembrane domains in the target immune cell membrane must be sufficient to allow the assembly and cell surface expression of the synthetic receptor and synthetic signaling modules.

TM refers to sequence of amino acids (usually between 15-25 amino acids, mostly hydrophobic) from a protein that forms a single alpha helix that is inserted in the lipid bilayer of a cell.

In an embodiment, the first positively charged amino acid and first negatively charged amino acid are positioned to allow electrostatic interactions between the first and second synthetic transmembrane domains in the target immune cell membrane, while avoiding electrostatic interactions with a native transmembrane domain from an immune receptor and/or native immune receptor signaling protein expressed by the target immune cell.

Positively charged (or basic) amino acids include lysine (K), arginine (R) and histidine (H). Preferably, the positively charged amino acid is arginine or lysine. In an embodiment, the positively charged amino acid is arginine. In another embodiment, the positively charged amino acid is lysine.

Negatively charged (or acidic) amino acids include aspartic acid (D) and glutamic acid (E). In an embodiment, the negatively charged amino acid is aspartic acid. In another embodiment, the negatively charged amino acid is glutamic acid.

The skilled person would understand that the first and second synthetic transmembrane domains may be variants of TMs of native (or endogenous) immune receptors, and are designed based on the sequences, and more particularly the position of the positively and negatively charged residues, of the native TMs. Table I below depicts the sequences of the predicted TMs of several human immune receptors (receptor and signaling modules), with the positively and negatively charged residues in capital letters.

TABLE I
Predicted TMs of several native human immune receptors
and associated immune receptor signaling proteins

| | | | |
|---|---|---|---|
| | | Predicted | |
| | | Tm | Sequence N- to C-terminal |
| Gene name | Type | location¹ | (SEQ ID NO:) |

Receptor (ligand-binding) modules

| | | | |
|---|---|---|---|
| T cell receptor delta constant (TRDC) | 1 | 130:152 | vlglRmlfaKtvavnflltaKlff (1) |
| T cell receptor alpha constant (TRAC) | 1 | 116:138 | vigfRilllKvagfnllmtlRlw (2) |
| T cell receptor beta constant 1 (TRBC1) | 1 | 150:171 | illgKatlyavlvsalvlmamv (3) |
| T cell receptor beta constant 2 (TRBC2) | 1 | 150:171 | illgKatlyavlvsalvlmamv (3) |
| T cell receptor gamma constant 1 | 1 | 139:161 | yymylllllKsvvyfaiitccll (4) |
| (TRGC1) | | | |
| T cell receptor gamma constant 2 | 1 | 155:177 | yytylllllKsvvyfaiitccll (5) |
| (TRGC2) | | | |
| Natural cytotoxicity triggering receptor 1 | 1 | 256:274 | llRmglaflvlvalvwflv (6) |
| (NCTR1) | | | |
| Natural cytotoxicity triggering receptor 2 | 1 | 193:213 | lvpvfcgllvaKslvlsallv (7) |
| (NCTR2) | | | |
| Natural cytotoxicity triggering receptor 3 | 1 | 136:156 | agtvlllRagfyavsflsvav (8) |
| (NCTR3) | | | |
| Killer cell immunoglobulin-like receptor | 1 | 243:263 | aviRysvaiilftilpffllh (9) |
| 2DL4 (KI2L4) | | | |
| NKG2-F type II integral membrane | 2 | 75:95 | vlgiicivlmatvlKtivlip (10) |
| protein (NKG2F) | | | |
| NKG2-E type II integral membrane | 2 | 71:93 | ltaEvlgiicivlmatvlKtivl (11) |
| protein (NKG2E) | | | |
| NKG2-D type II integral membrane | 2 | 52:72 | pfffccfiavamgiRfiimva (12) |
| protein (NKG2D) | | | |
| NKG2-C type II integral membrane | 2 | 71:93 | vlgiicivlmatvlKtivlipfl (13) |
| protein (NKG2C) | | | |
| Killer cell immunoglobulin-like receptor | 1 | 246:264 | vligtsvvKipftillffl (14) |
| 2DS1 (KI2S1) | | | |
| Killer cell immunoglobulin-like receptor | 1 | 246:265 | vligtsvvKipftillffll (15) |
| 2DS2 (KI2S2) | | | |
| Killer cell immunoglobulin-like receptor | 1 | 246:264 | vligtsvvKipftillffl (16) |
| 2DS3 (KI2S3) | | | |
| Killer cell immunoglobulin-like receptor | 1 | 246:265 | vligtsvvKipftillffll (15) |
| 2DS4 (KI2S4) | | | |
| Killer cell immunoglobulin-like receptor | 1 | 246:264 | vligtsvvKipftillffl (16) |
| 2DS5 (KI2S5) | | | |
| Killer cell immunoglobulin-like receptor | 1 | 341:360 | iligtsvvKipftillffll (17) |
| 3DS1 (KI3S1) | | | |
| Triggering receptor expressed on myeloid | 1 | 206:226 | ivillaggflsKslvfsvlfa (18) |
| cells 1 (TREM1) | | | |
| Triggering receptor expressed on myeloid | 1 | 175:195 | illllacifliKilaasalwa (19) |
| cells 2 (TREM2) | | | |
| Platelet glycoprotein VI (GPVI) | 1 | 268:288 | gnlvRiclgaviliilagfla (20) |

Signaling modules

| | | | |
|---|---|---|---|
| T-cell surface glycoprotein CD3 delta | 1 | 106:126 | giivtDviatlllalgvfcfa (21) |
| chain (CD3D) | | | |
| T-cell surface glycoprotein CD3 epsilon | 1 | 127:152 | vsvativivDicitggllllvyyws (22) |
| chain (CD3E) | | | |
| T-cell surface glycoprotein CD3 gamma | 1 | 117:137 | gflfaEivsifvlavgvyfia (23) |
| chain (CD3G) | | | |
| T-cell surface glycoprotein CD3 zeta | 1 | 31:51 | lcyllDgilfiygviltalfl (24) |
| chain (CD3Z) | | | |
| Hematopoietic cell signal transducer | 1 | 49:69 | llaglvaaDavasllivgavf (25) |
| (HCST or DAP10) | | | |
| TYRO protein tyrosine kinase-binding | 1 | 41:61 | gvlagivmgDlvltvlialav (26) |
| protein (TYROBP or DAP12) | | | |
| B-cell antigen receptor complex- | 1 | 144:165 | iitaEgiillfcavvpgtlllf (27) |
| associated protein alpha chain (CD79A) | | | |
| High affinity immunoglobulin epsilon | 1 | 24:44 | lcyilDailflygivltllyc (28) |
| receptor subunit gamma (FCERG) | | | |
| High affinity immunoglobulin epsilon | 1 | 206:224 | ffipllvvilfavDtglfi (29) |
| receptor subunit alpha (FCERA) | | | |
| Low affinity immunoglobulin gamma Fc | 1 | 209:229 | vsfclvmvllfavDtglyfsv (30) |
| region receptor III-A (FCG3A) | | | |
| Fc receptor-like protein 1 (FCRL1) | 1 | 308:328 | gviEgllstlgpatvallfcy (31) |

¹According to UniProt, TMHMM server 2.0, TMpred (ExPASy) and/or PSIPRED

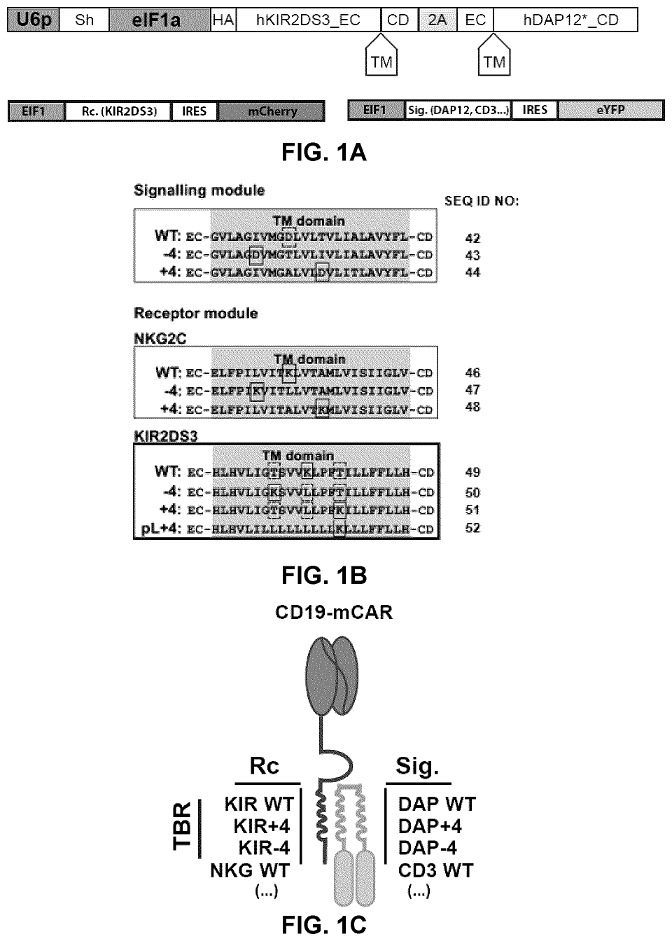

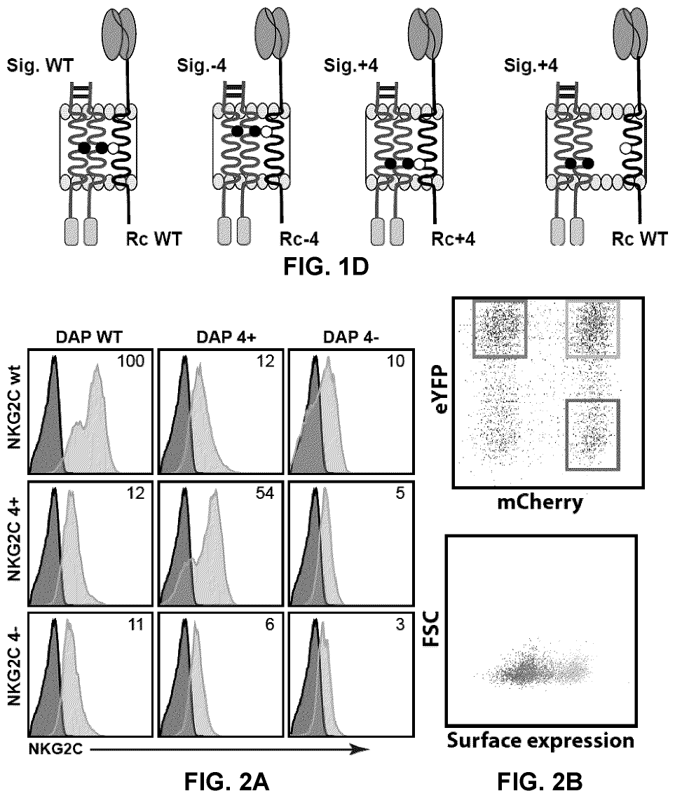

The chimeric receptor assembly according to the present disclosure will be illustrated taking the TMs of KI2S1 and TYROBP as representative examples of TMs of a receptor (ligand-binding) and signaling modules, respectively. The sequence of the native TMs of KI2S1 is vligtsvvKipftillffl, with the positively charged arginine residue at position 9 (starting from the extracellular domain). The sequence of the native TMs of TYROBP is gvlagivmgDlvltvlialav, with the negatively charged aspartic acid residue at position 10 (starting from the extracellular domain). In the plasma membrane of an immune cell (NK cell), the positively charged arginine residue of KI2S1 is able to form electrostatic interactions with negatively charged aspartic acid residues of TYROBP (in homodimeric form) as they are found in the same area of the plasma membrane within the TM helixes (see FIG. 1D, left scheme). The modular chimeric receptor according to the present disclosure is designed by moving the position of the positively and negatively charged residues up or down within the TM helixes (preferably by 3-5, more preferably by 4 residues in view of the fact that alpha helices have in average ˜3.6 residues per turn) in the ligand-binding and signaling modules in order to change the binding registry. The rearranged ligand-binding and signaling modules are able to interact within the plasma membrane (PM) because of the proper alignment of the positively and negatively charged residues allowing electrostatic interactions (see FIG. 1D, middle schemes, Sig.−4/Rc−4 and Sig.+4/Rc+4), but cannot assemble with native ligand-binding or signaling modules since the positively and negatively charged residues are not aligned in the PM (thereby leading to no or very weak electrostatic interactions) (FIG. 1D, right scheme). It would be understood that this approach of moving the position of the of the positively and negatively charged residues up or down within the TM helixes to promote exclusive assembly of modular chimeric receptor components may be applied to any immune receptor TM and signaling/accessory protein TM. In an embodiment, the positively and negatively charged residues are not located in the first 3 positions at the N- or C-terminal end of the synthetic transmembrane domain.

The present disclosure thus relates to variants of the TM of the above-identified immune receptors or signaling/accessory proteins in which the positively- or negatively-charged residue is moved at least 3 positions (e.g., 3, 4, 5, 6, . . . ) and preferably at least 4 positions, N or C-terminal relative to their position in the native TM. In an embodiment, the positively- or negatively-charged residue are moved at least 3-5 (preferably 4), 7-9 (preferably 8) and/or 11-13 (preferably 12) positions N or C-terminal relative to their position in the native TM. It should be understood that the TM variants may comprise more than one positively- or negatively-charged residues to allow the formation of a chimeric receptor assembly comprising several receptor modules and/or signaling modules (as exemplified with the assembly of the T cell receptor). For example, the first synthetic transmembrane domain (receptor module) may comprise 2 positively-charged residues, thereby allowing assembly with two different signaling/accessory modules, each comprising a negatively-charged residue in their TM properly positioned to allow electrostatic interactions with the positively-charged residues in the synthetic transmembrane domain of the receptor module.

It may be understood by the skilled person that the first and second synthetic transmembrane domains may be completely artificial, i.e. not derived from native transmembrane domains of known receptors or signaling/accessory proteins, which may be designed in silico based on the knowledge of transmembrane domain structures and sequences. Such artificial transmembrane domain may be identified or designed using transmembrane prediction tools such as TMpred (K. Hofmann & W. Stoffel (1993) TMbase—A database of membrane spanning proteins segments Biol. Chem. Hoppe-Seyler 374,166), TMHMM (A. Krogh, B. Larsson, G. von Heijne, and E. L. L. Sonnhammer. Predicting transmembrane protein topology with a hidden Markov model: Application to complete genomes. Journal of Molecular Biology, 305(3):567-580, January 2001. The first and second synthetic transmembrane domains may also be variants of known receptors or signaling/accessory proteins designed by introducing targeted mutations in the sequence of the native transmembrane domains. Such mutations include at least moving the position of the positively- and negatively-charged residues within the TM helixes, as noted above, but may also include other mutations to stabilize, or give a desired configuration to, the TM helix, for example.

First Synthetic Transmembrane Domains

In an embodiment, the first synthetic transmembrane domain is a variant of the TM of TRDC and comprises a sequence having at least 40% or 50% identity with the sequence VLGLRMLFAKTVAVNFLLTAKLFF (SEQ ID NO:1), wherein the K residue at position 10 and/or the K residue at position 21 is/are replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the A residue at position 13, the V residue at position 14, or the N residue at position 15 is replaced by a positively charged amino acid; or
(ii) at least one of: the F residue at position 16, the L residue at position 17, or the L residue at position 18 is replaced by a positively charged amino acid.

In an embodiment, the first synthetic transmembrane domain comprises a sequence having at least 60%, 70%, 80% or 90% identity with the sequence VLGLRMLFAKTVAVNFLLTAKLFF.

In an embodiment, the V residue at position 14 of SEQ ID NO:1 is replaced by a positively charged amino acid. In an embodiment, the L residue at position 17 of SEQ ID NO:1 is replaced by a positively charged amino acid. In an embodiment, the amino acid(s) located 4 residues N-terminal and/or C-terminal of the positively charged amino acid is/are threonine. In an embodiment, one or more of the residues at positions 10, 13, 18 and 21 of SEQ ID NO:1 are threonine residues. In an embodiment, at least 1, 2, 3, 4, 5, 6, 7 or 8 of the residues in SEQ ID NO:1 are replaced by leucine residues.

In an embodiment, the first synthetic transmembrane domain is a variant of the TM of TRAC and comprises a sequence having at least 40% or 50% identity with the sequence VIGFRILLLKVAGFNLLMTLRLW (SEQ ID NO:2), wherein the R residue at position 5, the K residue at position 10 and/or the R residue at position 21 is/are replaced by an uncharged amino acid, preferably a hydrophobic amino acid, and wherein at least one of the other residues located at least 3 positions from the above-noted residue(s) is replaced by a positively charged amino acid.

In an embodiment:
(i) at least one of: the L residue at position 8 or the L residue at position 9 is replaced by a positively charged amino acid;
(ii) at least one of: the I residue at position 6 or the L residue at position 7 is replaced by a positively charged amino acid;
(iii) at least one of: the G residue at position 13, the F residue at position 14, or the N residue at position 15 is replaced by a positively charged amino acid; and/or
(iv) at least one of: the L residue at position 16, the L residue at position 17, or the M residue at position 18 is replaced by a positively charged amino acid.

In an embodiment, the first synthetic transmembrane domain comprises a sequence having at least 60%, 70%, 80% or 90% identity with the sequence VIGFRILLLKVAGFNLLMTLRLW.

In an embodiment, the L residue at position 9 of SEQ ID NO:2 is replaced by a positively charged amino acid. In an embodiment, the F residue at position 14 of SEQ ID NO:2 is replaced by a positively charged amino acid. In an embodiment, the L residue at position 17 of SEQ ID NO:2 is replaced by a positively charged amino acid. In an embodiment, the amino acid(s) located 4 residues N-terminal and/or C-terminal of the positively charged amino acid is/are threonine. In an embodiment, one or more of the residues at positions 5, 10, 13, 18 and 21 of SEQ ID NO:2 are threonine residues. In an embodiment, at least 1, 2, 3, 4, 5, 6, 7 or 8 of the residues in SEQ ID NO:2 are replaced by leucine residues.

In an embodiment, the first synthetic transmembrane domain is a variant of the TM of TRBC1 or TRBC2 and comprises a sequence having at least 40% or 50% identity with the sequence ILLGKATLYAVLVSALVLMAMV (SEQ ID NO:3), wherein the K residue at position 5 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the L residue at position 8, the Y residue at position 9, or the A residue at position 10 is replaced by a positively charged amino acid;
(ii) at least one of: the L residue at position 12, the V residue at position 13, or the S residue at position 14 is replaced by a positively charged amino acid; and/or
(iii) at least one of: the L residue at position 16, the V residue at position 17, or the L residue at position 18 is replaced by a positively charged amino acid.

In an embodiment, the first synthetic transmembrane domain comprises a sequence having at least 60%, 70%, 80% or 90% identity with the sequence ILLGKATLYAVLVSALVLMAMV.

In an embodiment, the Y residue at position 9 of SEQ ID NO:3 is replaced by a positively charged amino acid. In an embodiment, the V residue at position 13 of SEQ ID NO:3 is replaced by a positively charged amino acid. In an embodiment, the V residue at position 17 of SEQ ID NO:3 is replaced by a positively charged amino acid. In an embodiment, the amino acid(s) located 4 residues N-terminal and/or C-terminal of the positively charged amino acid is/are threonine. In an embodiment, one or more of the residues at positions 5, 9, 13, 17 and 21 of SEQ ID NO:3 are threonine residues. In an embodiment, at least 1, 2, 3, 4, 5, 6, 7 or 8 of the residues in SEQ ID NO:3 are replaced by leucine residues.

In an embodiment, the first synthetic transmembrane domain is a variant of the TM of TRGC1 and comprises a sequence having at least 40% or 50% identity with the sequence YYMYLLLLLKSVVYFAIITCCLL (SEQ ID NO:4), wherein the K residue at position 10 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the L residue at position 5, the L residue at position 6, or the L residue at position 7 is replaced by a positively charged amino acid;
(ii) at least one of: the V residue at position 13, the Y residue at position 14, or the F residue at position 15 is replaced by a positively charged amino acid; and/or
(iii) at least one of: the I residue at position 17, the I residue at position 18, or the T residue at position 19 is replaced by a positively charged amino acid.

In an embodiment, the first synthetic transmembrane domain comprises a sequence having at least 60%, 70%, 80% or 90% identity with the sequence YYMYLLLLLKSVVYFAIITCCLL.

In an embodiment, the L residue at position 6 of SEQ ID NO:4 is replaced by a positively charged amino acid. In an embodiment, the Y residue at position 14 of SEQ ID NO:4 is replaced by a positively charged amino acid. In an embodiment, the I residue at position 18 of SEQ ID NO:4 is replaced by a positively charged amino acid. In an embodiment, the amino acid(s) located 4 residues N-terminal and/or C-terminal of the positively charged amino acid is/are threonine. In an embodiment, one or more of the residues at positions 2, 10, 14 and 18 of SEQ ID NO:4 are threonine residues. In an embodiment, at least 1, 2, 3, 4, 5, 6, 7 or 8 of the residues in SEQ ID NO:4 are replaced by leucine residues.

In an embodiment, the first synthetic transmembrane domain is a variant of the TM of TRGC2 and comprises a sequence having at least 40% or 50% identity with the sequence YYTYLLLLLKSVVYFAIITCCLL (SEQ ID NO:5), wherein the K residue at position 10 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the L residue at position 5, the L residue at position 6, or the L residue at position 7 is replaced by a positively charged amino acid;
(ii) at least one of: the V residue at position 13, the Y residue at position 14, or the F residue at position 15 is replaced by a positively charged amino acid; and/or
(iii) at least one of: the I residue at position 17, the I residue at position 18, or the T residue at position 19 is replaced by a positively charged amino acid.

In an embodiment, the first synthetic transmembrane domain comprises a sequence having at least 60%, 70%, 80% or 90% identity with the sequence YYTYLLLLLKSVVYFAIITCCLL.

In an embodiment, the L residue at position 6 of SEQ ID NO:5 is replaced by a positively charged amino acid. In an embodiment, the Y residue at position 14 of SEQ ID NO:5 is replaced by a positively charged amino acid. In an embodiment, the I residue at position 18 of SEQ ID NO:5 is replaced by a positively charged amino acid. In an embodiment, the amino acid(s) located 4 residues N-terminal and/or C-terminal of the positively charged amino acid is/are threonine. In an embodiment, one or more of the residues at positions 2, 10, 14 and 18 of SEQ ID NO:5 are threonine residues. In an embodiment, at least 1, 2, 3, 4, 5, 6, 7 or 8 of the residues in SEQ ID NO:5 are replaced by leucine residues.

In an embodiment, the first synthetic transmembrane domain is a variant of the TM of NCTR1 and comprises a sequence having at least 40% or 50% identity with the sequence LLRMGLAFLVLVALVWFLV (SEQ ID NO:6), wherein the R residue at position 3 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the L residue at position 6, the A residue at position 7, or the F residue at position 8 is replaced by a positively charged amino acid;
(ii) at least one of: the V residue at position 10, the L residue at position 11, or the V residue at position 12 is replaced by a positively charged amino acid; and/or
(iii) at least one of: the V residue at position 14, the W residue at position 15, or the F residue at position 16 is replaced by a positively charged amino acid.

In an embodiment, the first synthetic transmembrane domain comprises a sequence having at least 60%, 70%, 80% or 90% identity with the sequence LLRMGLAFLVLVALVWFLV.

In an embodiment, the A residue at position 7 of SEQ ID NO:6 is replaced by a positively charged amino acid. In an embodiment, the L residue at position 11 of SEQ ID NO:6 is replaced by a positively charged amino acid. In an embodiment, the W residue at position 15 of SEQ ID NO:6 is replaced by a positively charged amino acid. In an embodiment, the amino acid(s) located 4 residues N-terminal and/or C-terminal of the positively charged amino acid is/are threonine. In an embodiment, one or more of the residues at positions 7, 11, 15 and 19 of SEQ ID NO:6 are threonine residues. In an embodiment, at least 1, 2, 3, 4, 5, 6, 7 or 8 of the residues in SEQ ID NO:6 are replaced by leucine residues.

In an embodiment, the first synthetic transmembrane domain is a variant of the TM of NCTR2 and comprises a sequence having at least 40% or 50% identity with the sequence LVPVFCGLLVAKSLVLSALLV (SEQ ID NO:7), wherein the K residue at position 12 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the G residue at position 7, the L residue at position 8, or the L residue at position 9 is replaced by a positively charged amino acid; and/or
(ii) at least one of: the V residue at position 11, the L residue at position 12, or the S residue at position 13 is replaced by a positively charged amino acid.

In an embodiment, the first synthetic transmembrane domain comprises a sequence having at least 60%, 70%, 80% or 90% identity with the sequence LVPVFCGLLVAKSLVLSALLV.

In an embodiment, the L residue at position 8 of SEQ ID NO:7 is replaced by a positively charged amino acid. In an embodiment, the L residue at position 12 of SEQ ID NO:7 is replaced by a positively charged amino acid. In an embodiment, the amino acid(s) located 4 residues N-terminal and/or C-terminal of the positively charged amino acid is/are threonine. In an embodiment, one or more of the residues at positions 4, 8, 12 and 16 of SEQ ID NO:7 are threonine residues. In an embodiment, at least 1, 2, 3, 4, 5, 6, 7 or 8 of the residues in SEQ ID NO:7 are replaced by leucine residues.

In an embodiment, the first synthetic transmembrane domain is a variant of the TM of NCTR3 and comprises a sequence having at least 40% or 50% identity with the sequence AGTVLLLRAGFYAVSFLSVAV (SEQ ID NO:8), wherein the R residue at position 8 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the T residue at position 3, the V residue at position 4, or the L residue at position 5 is replaced by a positively charged amino acid;
(ii) at least one of: the F residue at position 11, the Y residue at position 12, or the A residue at position 13 is replaced by a positively charged amino acid; and/or
(iii) at least one of: the S residue at position 15, the F residue at position 16, or the L residue at position 17 is replaced by a positively charged amino acid.

In an embodiment, the first synthetic transmembrane domain comprises a sequence having at least 60%, 70%, 80% or 90% identity with the sequence LVPVFCGLLVAKSLVLSALLV.

In an embodiment, the Y residue at position 12 of SEQ ID NO:8 is replaced by a positively charged amino acid. In an embodiment, the F residue at position 16 of SEQ ID NO:8 is replaced by a positively charged amino acid. In an embodiment, the amino acid(s) located 4 residues N-terminal and/or C-terminal of the positively charged amino acid is/are threonine. In an embodiment, one or more of the residues at positions 4, 8, 12, 16 and 20 of SEQ ID NO:8 are threonine residues. In an embodiment, at least 1, 2, 3, 4, 5, 6, 7 or 8 of the residues in SEQ ID NO:8 are replaced by leucine residues.

In an embodiment, the first synthetic transmembrane domain is a variant of the TM of KI2L4 and comprises a sequence having at least 40% or 50% identity with the sequence AVIRYSVAIILFTILPFFLLH (SEQ ID NO:9), wherein the R residue at position 4 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the V residue at position 7, the A residue at position 8, or the I residue at position 9 is replaced by a positively charged amino acid;
(ii) at least one of: the L residue at position 11, the F residue at position 12, or the T residue at position 13 is replaced by a positively charged amino acid; and/or
(iii) at least one of: the L residue at position 15, the P residue at position 16, or the F residue at position 18 is replaced by a positively charged amino acid.

In an embodiment, the first synthetic transmembrane domain comprises a sequence having at least 60%, 70%, 80% or 90% identity with the sequence AVIRYSVAIILFTILPFFLLH.

In an embodiment, the A residue at position 8 of SEQ ID NO:9 is replaced by a positively charged amino acid. In an embodiment, the T residue at position 12 of SEQ ID NO:9 is replaced by a positively charged amino acid. In an embodiment, the P residue at position 16 of SEQ ID NO:9 is replaced by a positively charged amino acid. In an embodiment, the amino acid(s) located 4 residues N-terminal and/or C-terminal of the positively charged amino acid is/are threonine. In an embodiment, one or more of the residues at positions 4, 8, 12, 16 and 20 of SEQ ID NO:9 are threonine residues. In an embodiment, at least 1, 2, 3, 4, 5, 6, 7 or 8 of the residues in SEQ ID NO:9 are replaced by leucine residues.

In an embodiment, the first synthetic transmembrane domain is a variant of the TM of NKG2F and comprises a sequence having at least 40% or 50% identity with the sequence VLGIICIVLMATVLKTIVLIP (SEQ ID NO:10), wherein the K residue at position 15 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the M residue at position 10, the A residue at position 11, or the T residue at position 12 is replaced by a positively charged amino acid; and/or
(ii) at least one of: the C residue at position 6, the I residue at position 7, or the V residue at position 8 is replaced by a positively charged amino acid.

In an embodiment, the first synthetic transmembrane domain comprises a sequence having at least 60%, 70%, 80% or 90% identity with the sequence VLGIICIVLMATVLKTIVLIP.

In an embodiment, the A residue at position 11 of SEQ ID NO:10 is replaced by a positively charged amino acid. In an embodiment, the I residue at position 7 of SEQ ID NO:10 is replaced by a positively charged amino acid. In an embodiment, the amino acid(s) located 4 residues N-terminal and/or C-terminal of the positively charged amino acid is/are threonine. In an embodiment, one or more of the residues at positions 3, 7, 11 and 15 of SEQ ID NO:10 are threonine residues. In an embodiment, at least 1, 2, 3, 4, 5, 6, 7 or 8 of the residues in SEQ ID NO:10 are replaced by leucine residues.

In an embodiment, the first synthetic transmembrane domain is a variant of the TM of NKG2E and comprises a sequence having at least 40% or 50% identity with the sequence LTAEVLGIICIVLMATVLKTIVL (SEQ ID NO:11), wherein the K residue at position 19 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the M residue at position 14, the A residue at position 15, or the T residue at position 16 is replaced by a positively charged amino acid;
(ii) at least one of: the C residue at position 10, the I residue at position 11, or the V residue at position 12 is replaced by a positively charged amino acid; and/or
(iii) at least one of: the L residue at position 6, the G residue at position 7, or the I residue at position 8 is replaced by a positively charged amino acid.

In an embodiment, the first synthetic transmembrane domain comprises a sequence having at least 60%, 70%, 80% or 90% identity with the sequence LTAEVLGIICIVLMATVLKTIVL.

In an embodiment, the A residue at position 15 of SEQ ID NO:11 is replaced by a positively charged amino acid. In an embodiment, the I residue at position 11 of SEQ ID NO:11 is replaced by a positively charged amino acid. In an embodiment, the G residue at position 7 of SEQ ID NO:12 is replaced by a positively charged amino acid. In an embodiment, the amino acid(s) located 4 residues N-terminal and/or C-terminal of the positively charged amino acid is/are threonine. In an embodiment, one or more of the residues at positions 3, 7, 11, 15 and 19 of SEQ ID NO: 11 are threonine residues. In an embodiment, at least 1, 2, 3, 4, 5, 6, 7 or 8 of the residues in SEQ ID NO:11 are replaced by leucine residues.

In an embodiment, the first synthetic transmembrane domain is a variant of the TM of NKG2D and comprises a sequence having at least 40% or 50% identity with the sequence PFFFCCFIAVAMGIRFIIMVA (SEQ ID NO:12), wherein the R residue at position 15 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the V residue at position 10, the A residue at position 11, or the M residue at position 12 is replaced by a positively charged amino acid; and/or
(ii) at least one of: the C residue at position 6, the F residue at position 7, or the I residue at position 8 is replaced by a positively charged amino acid.

In an embodiment, the first synthetic transmembrane domain comprises a sequence having at least 60%, 70%, 80% or 90% identity with the sequence PFFFCCFIAVAMGIRFIIMVA.

In an embodiment, the A residue at position 11 of SEQ ID NO:12 is replaced by a positively charged amino acid. In an embodiment, the F residue at position 7 of SEQ ID NO:12 is replaced by a positively charged amino acid. In an embodiment, the amino acid(s) located 4 residues N-terminal and/or C-terminal of the positively charged amino acid is/are threonine. In an embodiment, one or more of the residues at positions 3, 7, 11 and 15 of SEQ ID NO:12 are threonine residues. In an embodiment, at least 1, 2, 3, 4, 5, 6, 7 or 8 of the residues in SEQ ID NO:12 are replaced by leucine residues.

In an embodiment, the first synthetic transmembrane domain is a variant of the TM of NKG2C and comprises a sequence having at least 40% or 50% identity with the sequence VLGIICIVLMATVLKTIVLIPFL (SEQ ID NO:13), wherein the K residue at position 15 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the M residue at position 10, the A residue at position 11, or the T residue at position 12 is replaced by a positively charged amino acid; and/or
(ii) at least one of: the C residue at position 6, the I residue at position 7, or the V residue at position 8 is replaced by a positively charged amino acid.

In an embodiment, the first synthetic transmembrane domain comprises a sequence having at least 60%, 70%, 80% or 90% identity with the sequence VLGIICIVLMATVLKTIVLIPFL.

In an embodiment, the A residue at position 11 of SEQ ID NO:13 is replaced by a positively charged amino acid. In an embodiment, the I residue at position 7 of SEQ ID NO:13 is replaced by a positively charged amino acid. In an embodiment, the amino acid(s) located 4 residues N-terminal and/or C-terminal of the positively charged amino acid is/are threonine. In an embodiment, one or more of the residues at positions 3, 7, 11 and 15 of SEQ ID NO:13 are threonine residues. In an embodiment, at least 1, 2, 3, 4, 5, 6, 7 or 8 of the residues in SEQ ID NO:13 are replaced by leucine residues. In an embodiment, the first synthetic transmembrane domain comprises the sequence VLGIICIVLMKTVLATIVLIPFL (SEQ ID NO:48).

In an embodiment, the first synthetic transmembrane domain is a variant of the TM of KI2S1 and comprises a sequence having at least 40% or 50% identity with the sequence VLIGTSWKIPFTILLFFL (SEQ ID NO:14), wherein the K residue at position 9 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the G residue at position 4, the T residue at position 5, or the S residue at position 6 is replaced by a positively charged amino acid; and/or
(ii) at least one of: the F residue at position 12, the T residue at position 13, or the I residue at position 14 is replaced by a positively charged amino acid.

In an embodiment, the first synthetic transmembrane domain comprises a sequence having at least 60%, 70%, 80% or 90% identity with the sequence VLIGTSVVKIPFTILLFFL.

In an embodiment, the T residue at position 5 of SEQ ID NO:14 is replaced by a positively charged amino acid. In an embodiment, the T residue at position 13 of SEQ ID NO:14 is replaced by a positively charged amino acid. In an embodiment, the amino acid(s) located 4 residues N-terminal and/or C-terminal of the positively charged amino acid is/are threonine. In an embodiment, one or more of the residues at positions 1, 9, 13 and 18 of SEQ ID NO:14 are threonine residues. In an embodiment, at least 1, 2, 3, 4, 5, 6, 7 or 8 of the residues in SEQ ID NO:15 are replaced by leucine residues.

In an embodiment, the first synthetic transmembrane domain is a variant of the TM of KI2S2 or KI2S4 and comprises a sequence having at least 40% or 50% identity with the sequence VLIGTSWKIPFTILLFFLL (SEQ ID NO:15), wherein the K residue at position 9 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the G residue at position 4, the T residue at position 5, or the S residue at position 6 is replaced by a positively charged amino acid; and/or
(ii) at least one of: the F residue at position 12, the T residue at position 13, or the I residue at position 14 is replaced by a positively charged amino acid.

In an embodiment, the first synthetic transmembrane domain comprises a sequence having at least 60%, 70%, 80% or 90% identity with the sequence VLIGTSVVKIPFTILLFFLL.

In an embodiment, the T residue at position 5 of SEQ ID NO:15 is replaced by a positively charged amino acid. In an embodiment, the T residue at position 13 of SEQ ID NO:15 is replaced by a positively charged amino acid. In an embodiment, the amino acid(s) located 4 residues N-terminal and/or C-terminal of the positively charged amino acid is/are threonine. In an embodiment, one or more of the residues at positions 1, 9, 13 and 17 of SEQ ID NO:15 are threonine residues. In an embodiment, at least 1, 2, 3, 4, 5, 6, 7 or 8 of the residues in SEQ ID NO:15 are replaced by leucine residues.

In an embodiment, the first synthetic transmembrane domain is a variant of the TM of KI2S3 or KI2S5 and comprises a sequence having at least 40% or 50% identity with the sequence VLIGTSWKLPFTILLFFL (SEQ ID NO:16), wherein the K residue at position 9 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the G residue at position 4, the T residue at position 5, or the S residue at position 6 is replaced by a positively charged amino acid; and/or
(ii) at least one of: the F residue at position 12, the T residue at position 13, or the I residue at position 14 is replaced by a positively charged amino acid.

In an embodiment, the first synthetic transmembrane domain comprises a sequence having at least 60%, 70%, 80% or 90% identity with the sequence VLIGTSVVKLPFTILLFFL.

In an embodiment, the T residue at position 5 of SEQ ID NO:16 is replaced by a positively charged amino acid. In an embodiment, the T residue at position 13 of SEQ ID NO:16 is replaced by a positively charged amino acid. In an embodiment, the amino acid(s) located 4 residues N-terminal and/or C-terminal of the positively charged amino acid is/are threonine. In an embodiment, one or more of the residues at positions 1, 9, 13 and 17 of SEQ ID NO:16 are threonine residues. In an embodiment, at least 1, 2, 3, 4, 5, 6, 7 or 8 of the residues in SEQ ID NO:16 are replaced by leucine residues. In an embodiment, the first synthetic transmembrane domain comprises the amino acid sequence VLIGTSVVLLPFKILLFFLL (SEQ ID NO:32), VLIILLVGTSVVKLLLFFLL (SEQ ID NO:33), VLIGTSVVTLPFKILLFFLL (SEQ ID NO:34), VLILLLLLLLLLKLLLFFLL (SEQ ID NO:35), VLILLLLGLLLLKLLLFFLL (SEQ ID NO:36), VLILLLLLALLLKLLLFFLL (SEQ ID NO:37) or VLILLLLLTLLLKLLLFFLL (SEQ ID NO:38). In a further embodiment, the first synthetic transmembrane domain comprises the amino acid sequence of SEQ ID NO:38.

In an embodiment, the first synthetic transmembrane domain is a variant of the TM of K13S1 and comprises a sequence having at least 40% or 50% identity with the sequence ILIGTSVVKIPFTILLFFLL (SEQ ID NO:17), wherein the K residue at position 9 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the G residue at position 4, the T residue at position 5, or the S residue at position 6 is replaced by a positively charged amino acid; and/or
(ii) at least one of: the F residue at position 12, the T residue at position 13, or the I residue at position 14 is replaced by a positively charged amino acid.

In an embodiment, the first synthetic transmembrane domain comprises a sequence having at least 60%, 70%, 80% or 90% identity with the sequence ILIGTSVVKIPFTILLFFLL.

In an embodiment, the T residue at position 5 of SEQ ID NO:17 is replaced by a positively charged amino acid. In an embodiment, the T residue at position 13 of SEQ ID NO:17 is replaced by a positively charged amino acid. In an embodiment, the amino acid(s) located 4 residues N-terminal and/or C-terminal of the positively charged amino acid is/are threonine. In an embodiment, one or more of the residues at positions 1, 9, 13 and 17 of SEQ ID NO:17 are threonine residues. In an embodiment, at least 1, 2, 3, 4, 5, 6, 7 or 8 of the residues in SEQ ID NO:17 are replaced by leucine residues.

In an embodiment, the first synthetic transmembrane domain is a variant of the TM of TREM1 and comprises a sequence having at least 40% or 50% identity with the sequence IVILLAGGFLSKSLVFSVLFA (SEQ ID NO:18), wherein the K residue at position 12 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the G residue at position 7, the G residue at position 8, or the F residue at position 9 is replaced by a positively charged amino acid; and/or
(ii) at least one of: the V residue at position 15, the F residue at position 16, or the S residue at position 17 is replaced by a positively charged amino acid.

In an embodiment, the first synthetic transmembrane domain comprises a sequence having at least 60%, 70%, 80% or 90% identity with the sequence IVILLAGGFLSKSLVFSVLFA.

In an embodiment, the G residue at position 8 of SEQ ID NO:18 is replaced by a positively charged amino acid. In an embodiment, the F residue at position 16 of SEQ ID NO:18 is replaced by a positively charged amino acid. In an embodiment, the amino acid(s) located 4 residues N-terminal and/or C-terminal of the positively charged amino acid is/are threonine. In an embodiment, one or more of the residues at positions 4, 12, and 20 of SEQ ID NO:18 are threonine residues. In an embodiment, at least 1, 2, 3, 4, 5, 6, 7 or 8 of the residues in SEQ ID NO:18 are replaced by leucine residues.

In an embodiment, the first synthetic transmembrane domain is a variant of the TM of TREM2 and comprises a sequence having at least 40% or 50% identity with the sequence ILLLLACIFLIKILAASALWA (SEQ ID NO:19), wherein the K residue at position 12 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the C residue at position 7, the I residue at position 8, or the F residue at position 9 is replaced by a positively charged amino acid; and/or
(ii) at least one of: the A residue at position 15, the A residue at position 16, or the S residue at position 17 is replaced by a positively charged amino acid.

In an embodiment, the first synthetic transmembrane domain comprises a sequence having at least 60%, 70%, 80% or 90% identity with the sequence ILLLLACIFLIKILAASALWA.

In an embodiment, the I residue at position 8 of SEQ ID NO:19 is replaced by a positively charged amino acid. In an embodiment, the A residue at position 16 of SEQ ID NO:19 is replaced by a positively charged amino acid. In an embodiment, the amino acid(s) located 4 residues N-terminal and/or C-terminal of the positively charged amino acid is/are threonine. In an embodiment, one or more of the residues at positions 4, 12, and 20 of SEQ ID NO:19 are threonine residues. In an embodiment, at least 1, 2, 3, 4, 5, 6, 7 or 8 of the residues in SEQ ID NO:19 are replaced by leucine residues.

In an embodiment, the first synthetic transmembrane domain is a variant of the TM of GPVI and comprises a sequence having at least 40% or 50% identity with the sequence GNLVRICLGAVILIILAGFLA (SEQ ID NO:20), wherein the R residue at position 5 is replaced by an uncharged amino acid, preferably a hydrophobic amino acid, and wherein at least one of the other residues located at least 3 positions from the above-noted residue(s) is/are replaced by a positively charged amino acid.

In an embodiment:
(i) at least one of: the L residue at position 8, the G residue at position 9, or the A residue at position 10 is replaced by a positively charged amino acid;
(ii) at least one of: the I residue at position 12, the L residue at position 13, or the I residue at position 14 is replaced by a positively charged amino acid; and/or
(iii) at least one of: the L residue at position 16, the A residue at position 17, or the G residue at position 18 is replaced by a positively charged amino acid.

In an embodiment, the first synthetic transmembrane domain comprises a sequence having at least 60%, 70%, 80% or 90% identity with the sequence GNLVRICLGAVILIILAGFLA.

In an embodiment, the G residue at position 9 of SEQ ID NO:20 is replaced by a positively charged amino acid. In an embodiment, the L residue at position 13 of SEQ ID NO:20 is replaced by a positively charged amino acid. In an embodiment, the A residue at position 17 of SEQ ID NO:20 is replaced by a positively charged amino acid. In an embodiment, the amino acid(s) located 4 residues N-terminal and/or C-terminal of the positively charged amino acid is/are threonine. In an embodiment, one or more of the residues at positions 5, 9, 13 and 17 of SEQ ID NO:20 are threonine residues. In an embodiment, at least 1, 2, 3, 4, 5, 6, 7 or 8 of the residues in SEQ ID NO:20 are replaced by leucine residues.

Second Synthetic Transmembrane Domains

In an embodiment, the second synthetic transmembrane domain is a variant of the TM of CD3D and comprises a sequence having at least 40% or 50% identity with the sequence GIIVTDVIATLLLALGVFCFA (SEQ ID NO:21), wherein the D residue at position 6 is replaced by an uncharged amino acid, preferably a hydrophobic amino acid, and wherein at least one of the other residues located at least 3 positions from the above-noted residue(s) is/are replaced by a negatively charged amino acid.

In an embodiment:
(i) at least one of: the A residue at position 9, the T residue at position 10, or the L residue at position 11 is replaced by a negatively charged amino acid; and/or
(ii) at least one of: the L residue at position 13, the A residue at position 14, or the L residue at position 15 is replaced by a negatively charged amino acid.

In an embodiment, the second synthetic transmembrane domain comprises a sequence having at least 60%, 70%, 80% or 90% identity with the sequence GIIVTDVIATLLLALGVFCFA.

In an embodiment, the T residue at position 10 of SEQ ID NO:21 is replaced by a negatively charged amino acid. In an embodiment, the A residue at position 14 of SEQ ID NO:21 is replaced by a negatively charged amino acid. In an embodiment, the amino acid(s) located 4 residues N-terminal and/or C-terminal of the negatively charged amino acid is/are threonine. In an embodiment, one or more of the residues at positions 6, 10, 14 and 18 of SEQ ID NO:21 are threonine residues. In an embodiment, at least 1, 2, 3, 4, 5, 6, 7 or 8 of the residues in SEQ ID NO:21 are replaced by leucine residues.

In an embodiment, the second synthetic transmembrane domain is a variant of the TM of CD3E and comprises a sequence having at least 40% or 50% identity with the sequence VSVATIVIVDICITGGLLLLVYYWS (SEQ ID NO:22), wherein the D residue at position 10 is replaced by an uncharged amino acid, preferably a hydrophobic amino acid, and wherein at least one of the other residues located at least 3 positions from the above-noted residue(s) is/are replaced by a negatively charged amino acid.

In an embodiment:
(i) at least one of: the T residue at position 5, the I residue at position 6, or the V residue at position 7 is replaced by a negatively charged amino acid;
(ii) at least one of: the I residue at position 13, the T residue at position 14, or the G residue at position 15 is replaced by a negatively charged amino acid; and/or
(iii) at least one of: the L residue at position 17, the L residue at position 18, or the L residue at position 19 is replaced by a negatively charged amino acid.

In an embodiment, the second synthetic transmembrane domain comprises a sequence having at least 60%, 70%, 80% or 90% identity with the sequence VSVATIVIVDICITGGLLLLVYYWS.

In an embodiment, the I residue at position 6 of SEQ ID NO:22 is replaced by a negatively charged amino acid. In an embodiment, the T residue at position 14 of SEQ ID NO:22 is replaced by a negatively charged amino acid. In an embodiment, the L residue at position 18 of SEQ ID NO:22 is replaced by a negatively charged amino acid. In an embodiment, the amino acid(s) located 4 residues N-terminal and/or C-terminal of the negatively charged amino acid is/are threonine. In an embodiment, one or more of the residues at positions 6, 10, 14 and 18 of SEQ ID NO:22 are threonine residues. In an embodiment, at least 1, 2, 3, 4, 5, 6, 7 or 8 of the residues in SEQ ID NO:22 are replaced by leucine residues.

In an embodiment, the second synthetic transmembrane domain is a variant of the TM of CD3G and comprises a sequence having at least 40% or 50% identity with the sequence GFLFAEIVSIFVLAVGVYFIA (SEQ ID NO:23), wherein the E residue at position 6 is replaced by an uncharged amino acid, preferably a hydrophobic amino acid, and wherein at least one of the other residues located at least 3 positions from the above-noted residue(s) is/are replaced by a negatively charged amino acid.

In an embodiment:
(i) at least one of: the S residue at position 9, the I residue at position 10, or the F residue at position 11 is replaced by a negatively charged amino acid; and/or
(ii) at least one of: the L residue at position 13, the A residue at position 14, or the V residue at position 15 is replaced by a negatively charged amino acid.

In an embodiment, the second synthetic transmembrane domain comprises a sequence having at least 60%, 70%, 80% or 90% identity with the sequence GFLFAEIVSIFVLAVGVYFIA.

In an embodiment, the I residue at position 10 of SEQ ID NO:23 is replaced by a negatively charged amino acid. In an embodiment, the A residue at position 14 of SEQ ID NO:23 is replaced by a negatively charged amino acid. In an embodiment, the amino acid(s) located 4 residues N-terminal and/or C-terminal of the negatively charged amino acid is/are threonine. In an embodiment, one or more of the residues at positions 6, 10, 14 and 18 of SEQ ID NO:23 are threonine residues. In an embodiment, at least 1, 2, 3, 4, 5, 6, 7 or 8 of the residues in SEQ ID NO:23 are replaced by leucine residues.

In an embodiment, the second synthetic transmembrane domain is a variant of the TM of CD3Z and comprises a sequence having at least 40% or 50% identity with the sequence LCYLLDGILFIYGVILTALFL (SEQ ID NO:24), wherein the D residue at position 6 is replaced by an uncharged amino acid, preferably a hydrophobic amino acid, and wherein at least one of the other residues located at least 3 positions from the above-noted residue(s) is/are replaced by a negatively charged amino acid.

In an embodiment:
(i) at least one of: the L residue at position 9, the F residue at position 10, or the I residue at position 11 is replaced by a negatively charged amino acid; and/or
(ii) at least one of: the G residue at position 13, the V residue at position 14, or the I residue at position 15 is replaced by a negatively charged amino acid.

In an embodiment, the second synthetic transmembrane domain comprises a sequence having at least 60%, 70%, 80% or 90% identity with the sequence LCYLLDGILFIYGVILTALFL. In an embodiment, the F residue at position 10 of SEQ ID NO:24 is replaced by a negatively charged amino acid. In an embodiment, the V residue at position 14 of SEQ ID NO:24 is replaced by a negatively charged amino acid. In an embodiment, the amino acid(s) located 4 residues N-terminal and/or C-terminal of the negatively charged amino acid is/are threonine. In an embodiment, one or more of the residues at positions 6, 10, 14 and 18 of SEQ ID NO:24 are threonine residues. In an embodiment, at least 1, 2, 3, 4, 5, 6, 7 or 8 of the residues in SEQ ID NO:24 are replaced by leucine residues.

In an embodiment, the second synthetic transmembrane domain is a variant of the TM of HCST/DAP10 and comprises a sequence having at least 40% or 50% identity with the sequence LLAGLVAADAVASLLIVGAVF (SEQ ID NO:25), wherein the D residue at position 9 is replaced by an uncharged amino acid, preferably a hydrophobic amino acid, and wherein at least one of the other residues located at least 3 positions from the above-noted residue(s) is/are replaced by a negatively charged amino acid.

In an embodiment:
(i) at least one of: the G residue at position 4, the L residue at position 5, or the V residue at position 6 is replaced by a negatively charged amino acid;
(ii) at least one of: the A residue at position 12, the S residue at position 13, or the L residue at position 14 is replaced by a negatively charged amino acid; and/or
(iii) at least one of: the I residue at position 16, the V residue at position 17, or the G residue at position 18 is replaced by a negatively charged amino acid.

In an embodiment, the second synthetic transmembrane domain comprises a sequence having at least 60%, 70%, 80% or 90% identity with the sequence LLAGLVAADAVASLLIVGAVF.

In an embodiment, the L residue at position 5 of SEQ ID NO:25 is replaced by a negatively charged amino acid. In an embodiment, the S residue at position 13 of SEQ ID NO:25 is replaced by a negatively charged amino acid. In an embodiment, the V residue at position 17 of SEQ ID NO:25 is replaced by a negatively charged amino acid. In an embodiment, the amino acid(s) located 4 residues N-terminal and/or C-terminal of the negatively charged amino acid is/are threonine. In an embodiment, one or more of the residues at positions 5, 9, 13 and 17 of SEQ ID NO:25 are threonine residues. In an embodiment, at least 1, 2, 3, 4, 5, 6, 7 or 8 of the residues in SEQ ID NO:25 are replaced by leucine residues.

In an embodiment, the second synthetic transmembrane domain is a variant of the TM of TYROBP/DAP12 and comprises a sequence having at least 40% or 50% identity with the sequence VLAGIVMGDLVLTVLIALAVYFL (SEQ ID NO:26), wherein the D residue at position 9 is replaced by an uncharged amino acid, preferably a hydrophobic amino acid, and wherein at least one of the other residues located at least 3 positions from the above-noted residue(s) is/are replaced by a negatively charged amino acid.

In an embodiment:
(i) at least one of: the G residue at position 4, the I residue at position 5, or the V residue at position 6 is replaced by a negatively charged amino acid;
(ii) at least one of: the L residue at position 12, the T residue at position 13, or the V residue at position 14 is replaced by a negatively charged amino acid; and/or
(iii) at least one of: the I residue at position 16, the A residue at position 17, or the L residue at position 18 is replaced by a negatively charged amino acid.

In an embodiment, the second synthetic transmembrane domain comprises a sequence having at least 60%, 70%, 80% or 90% identity with the sequence GVLAGIVMGDLVLTVLIALAV.

In an embodiment, the I residue at position 5 of SEQ ID NO:26 is replaced by a negatively charged amino acid. In an embodiment, the T residue at position 13 of SEQ ID NO:26 is replaced by a negatively charged amino acid. In an embodiment, the A residue at position 17 of SEQ ID NO:26 is replaced by a negatively charged amino acid. In an embodiment, the amino acid(s) located 4 residues N-terminal and/or C-terminal of the negatively charged amino acid is/are threonine. In an embodiment, one or more of the residues at positions 9, 13 and 21 of SEQ ID NO:26 are threonine residues. In an embodiment, at least 1, 2, 3, 4, 5, 6, 7 or 8 of the residues in SEQ ID NO:26 are replaced by leucine residues. In an embodiment, the second synthetic transmembrane domain comprises the sequence VLAGIVMGALVLDVLITLAVYFL (SEQ ID NO:39). In another embodiment, the second synthetic transmembrane domain comprises the sequence VLALAVLGIVMGDVLITLAVYFL (SEQ ID NO:40). In another embodiment, the second synthetic transmembrane domain comprises the sequence VLAGDVMGTLVLIVLIALAVYFL (SEQ ID NO:41).

In an embodiment, the second synthetic transmembrane domain is a variant of the TM of CD79A and comprises a sequence having at least 40% or 50% identity with the sequence IITAEGIILLFCAVVPGTLLLF (SEQ ID NO:27), wherein the E residue at position 5 is replaced by an uncharged amino acid, preferably a hydrophobic amino acid, and wherein at least one of the other residues located at least 3 positions from the above-noted residue(s) is/are replaced by a negatively charged amino acid.

In an embodiment:
(i) at least one of: the I residue at position 8, the L residue at position 9, or the L residue at position 10 is replaced by a negatively charged amino acid;
(ii) at least one of: the C residue at position 12, the A residue at position 13, or the V residue at position 14 is replaced by a negatively charged amino acid; and/or
(iii) at least one of: the G residue at position 16, the T residue at position 17, or the L residue at position 18 is replaced by a negatively charged amino acid.

In an embodiment, the second synthetic transmembrane domain comprises a sequence having at least 60%, 70%, 80% or 90% identity with the sequence IITAEGIILLFCAVVPGTLLLF.

In an embodiment, the L residue at position 9 of SEQ ID NO:27 is replaced by a negatively charged amino acid. In an embodiment, the A residue at position 13 of SEQ ID NO:27 is replaced by a negatively charged amino acid. In an embodiment, the T residue at position 17 of SEQ ID NO:27 is replaced by a negatively charged amino acid. In an embodiment, the amino acid(s) located 4 residues N-terminal and/or C-terminal of the negatively charged amino acid is/are threonine. In an embodiment, one or more of the residues at positions 5, 9, 13 and 17 of SEQ ID NO:27 are threonine residues. In an embodiment, at least 1, 2, 3, 4, 5, 6, 7 or 8 of the residues in SEQ ID NO:27 are replaced by leucine residues.

In an embodiment, the second synthetic transmembrane domain is a variant of the TM of FCERG and comprises a sequence having at least 40% or 50% identity with the sequence LCYILDAILFLYGIVLTLLYC (SEQ ID NO:28), wherein the D residue at position 6 is replaced by an uncharged amino acid, preferably a hydrophobic amino acid, and wherein at least one of the other residues located at least 3 positions from the above-noted residue(s) is/are replaced by a negatively charged amino acid.

In an embodiment:
(i) at least one of: the L residue at position 9, the F residue at position 10, or the L residue at position 11 is replaced by a negatively charged amino acid;
(ii) at least one of: the G residue at position 13, the I residue at position 14, or the V residue at position 15 is replaced by a negatively charged amino acid; and/or
(iii) at least one of: the T residue at position 17, the L residue at position 18, or the L residue at position 19 is replaced by a negatively charged amino acid.

In an embodiment, the second synthetic transmembrane domain comprises a sequence having at least 60%, 70%, 80% or 90% identity with the sequence LCYILDAILFLYGIVLTLLYC.

In an embodiment, the F residue at position 10 of SEQ ID NO:28 is replaced by a negatively charged amino acid. In an embodiment, the I residue at position 14 of SEQ ID NO:28 is replaced by a negatively charged amino acid. In an embodiment, the L residue at position 18 of SEQ ID NO:28 is replaced by a negatively charged amino acid. In an embodiment, the amino acid(s) located 4 residues N-terminal and/or C-terminal of the negatively charged amino acid is/are threonine. In an embodiment, one or more of the residues at positions 6, 10, 14 and 18 of SEQ ID NO:28 are threonine residues. In an embodiment, at least 1, 2, 3, 4, 5, 6, 7 or 8 of the residues in SEQ ID NO:28 are replaced by leucine residues.

In an embodiment, the second synthetic transmembrane domain is a variant of the TM of FCERA and comprises a sequence having at least 40% or 50% identity with the sequence FFIPLLVVILFAVDTGLFI (SEQ ID NO:29), wherein the D residue at position 14 is replaced by an uncharged amino acid, preferably a hydrophobic amino acid, and wherein at least one of the other residues located at least 3 positions from the above-noted residue(s) is/are replaced by a negatively charged amino acid.

In an embodiment:
(i) at least one of: the I residue at position 9, the L residue at position 10, or the F residue at position 11 is replaced by a negatively charged amino acid; and/or
(ii) at least one of: the L residue at position 5, the L residue at position 6, or the V residue at position 7 is replaced by a negatively charged amino acid.

In an embodiment, the second synthetic transmembrane domain comprises a sequence having at least 60%, 70%, 80% or 90% identity with the sequence FFIPLLVVILFAVDTGLFI.

In an embodiment, the L residue at position 10 of SEQ ID NO:29 is replaced by a negatively charged amino acid. In an embodiment, the L residue at position 6 of SEQ ID NO:29 is replaced by a negatively charged amino acid. In an embodiment, the amino acid(s) located 4 residues N-terminal and/or C-terminal of the negatively charged amino acid is/are threonine. In an embodiment, one or more of the residues at positions 6, 10 and 14 of SEQ ID NO:29 are threonine residues. In an embodiment, at least 1, 2, 3, 4, 5, 6, 7 or 8 of the residues in SEQ ID NO:29 are replaced by leucine residues.

In an embodiment, the second synthetic transmembrane domain is a variant of the TM of FCG3A and comprises a sequence having at least 40% or 50% identity with the sequence VSFCLVMVLLFAVDTGLYFSV (SEQ ID NO:30), wherein the D residue at position 14 is replaced by an uncharged amino acid, preferably a hydrophobic amino acid, and wherein at least one of the other residues located at least 3 positions from the above-noted residue(s) is/are replaced by a negatively charged amino acid.

In an embodiment:
(i) at least one of: the L residue at position 9, the L residue at position 10, or the F residue at position 11 is replaced by a negatively charged amino acid;
(ii) at least one of: the L residue at position 5, the V residue at position 6, or the M residue at position 7 is replaced by a negatively charged amino acid; and/or
(iii) at least one of: the L residue at position 17, the Y residue at position 18, or the F residue at position 19 is replaced by a negatively charged amino acid.

In an embodiment, the second synthetic transmembrane domain comprises a sequence having at least 60%, 70%, 80% or 90% identity with the sequence VSFCLVMVLLFAVDTGLYFSV.

In an embodiment, the L residue at position 10 of SEQ ID NO:30 is replaced by a negatively charged amino acid. In an embodiment, the V residue at position 6 of SEQ ID NO:30 is replaced by a negatively charged amino acid. In an embodiment, the amino acid(s) located 4 residues N-terminal and/or C-terminal of the negatively charged amino acid is/are threonine. In an embodiment, one or more of the residues at positions 6, 10 and 14 of SEQ ID NO:30 are threonine residues. In an embodiment, at least 1, 2, 3, 4, 5, 6, 7 or 8 of the residues in SEQ ID NO:30 are replaced by leucine residues.

In an embodiment, the second synthetic transmembrane domain is a variant of the TM of FCRL1 and comprises a sequence having at least 40% or 50% identity with the sequence GVIEGLLSTLGPATVALLFCY (SEQ ID NO:31), wherein the E residue at position 4 is replaced by an uncharged amino acid, preferably a hydrophobic amino acid, and wherein at least one of the other residues located at least 3 positions from the above-noted residue(s) is/are replaced by a negatively charged amino acid.

In an embodiment:
(i) at least one of: the L residue at position 7, the S residue at position 8, or the T residue at position 9 is replaced by a negatively charged amino acid;
(ii) at least one of: the G residue at position 11, the P residue at position 12, or the A residue at position 13 is replaced by a negatively charged amino acid; and/or
(iii) at least one of: the V residue at position 15, the A residue at position 16, or the L residue at position 17 is replaced by a negatively charged amino acid.

In an embodiment, the second synthetic transmembrane domain comprises a sequence having at least 60%, 70%, 80% or 90% identity with the sequence GVIEGLLSTLGPATVALLFCY.

In an embodiment, the S residue at position 8 of SEQ ID NO:31 is replaced by a negatively charged amino acid. In an embodiment, the P residue at position 12 of SEQ ID NO:31 is replaced by a negatively charged amino acid. In an embodiment, the amino acid(s) located 4 residues N-terminal and/or C-terminal of the negatively charged amino acid is/are threonine. In an embodiment, one or more of the residues at positions 4, 12, 16 and 20 of SEQ ID NO:31 are threonine residues. In an embodiment, at least 1, 2, 3, 4, 5, 6, 7 or 8 of the residues in SEQ ID NO:31 are replaced by leucine residues.

In an embodiment, the first synthetic transmembrane domain is a variant of the TM of KI2S3 (KIR2DS3) and the second synthetic transmembrane domain is a variant of the TM of DAP12 or CD3Z. In an embodiment, the first synthetic transmembrane domain is a variant of the TM of NKG2C and the second synthetic transmembrane domain is a variant of the TM of DAP12 or CD3Z.

In another embodiment, the first synthetic transmembrane domain is a variant of the TM of NKG2C and the second synthetic transmembrane domain is a variant of the TM of DAP12 or CD3Z. In an embodiment, the first synthetic transmembrane domain is a variant of the TM of NKG2C and the second synthetic transmembrane domain is a variant of the TM of DAP12 or CD3Z.

The term “uncharged amino acid” as used herein refers to an amino acid whose side chain does not contain any charge, and include the hydrophobic amino acids alanine (A), valine (V), leucine (L), isoleucine (I), methionine (M), phenylalanine (F), tryptophan (W) and tyrosine (Y), the polar amino acids asparagine (N), glutamine (Q), serine (S) and threonine (T), as well as the special amino acids glycine (G), proline (P) and cysteine (C).

The terms “synthetic” mean that the transmembrane domain is an artificial transmembrane domain that is not found in nature, i.e. is not a transmembrane domain found in a naturally occurring human protein.

Sequence identity between two amino acid sequences may be determined by comparing each position in the aligned sequences. A degree of identity between amino acid sequences is a function of the number of identical amino acid at positions shared by the sequences. As used herein, a given percentage of identity between sequences denotes the degree of sequence identity in optimally aligned sequences. Optimal alignment of sequences for comparisons of identity may be conducted using a variety of algorithms and sequence alignment tools, such as the local homology algorithm of Smith and Waterman, 1981, Adv. Appl. Math 2: 482, the homology alignment algorithm of Needleman and Wunsch, 1970, J. Mol. Biol. 48: 443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215: 403-10 (using the published default settings). Software/tools for performing BLAST analysis may be available through the National Center for Biotechnology Information. Other sequence alignment tools such as Needle, Stretcher, Clustal Omega and Kalign are available through the European Bioinformatics Institute (EMBL-EBI).

In an embodiment, the modular chimeric receptor comprises one receptor module comprising a TM with two positively-charged residues and two signaling modules, wherein the TM of each of the signaling modules comprises a negatively charged residue positioned to interact with one of the positively-charged residues in the TM of the receptor module.

In an embodiment, the extracellular domain fused to the first synthetic transmembrane is the extracellular domain of a receptor (e.g., a recombinant or chimeric receptor). In an embodiment, the extracellular domain fused to the first synthetic transmembrane is a ligand-binding domain of a chimeric antigen receptor (CAR).

In some embodiments, the ligand, such as an antigen, is a protein expressed on the surface of cells (e.g., tumor cells).

Exemplary recombinant receptors, including CARs and recombinant TCRs, as well as methods for engineering and introducing the receptors into cells, include those described, for example, in international patent application publication numbers WO 2000/14257, WO 2013/126726, WO 2012/129514, WO 2014/031687, WO 2013/166321, WO 2013/071154, WO 2013/123061, US patent application publication numbers US 2002/131960, US 2013/287748, US 2013/0149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol, 2012 October; 24(5): 633-39; Wu et al., Cancer, 2012 March 18(2): 160-75. In some embodiments, the genetically engineered antigen receptors include a CAR as described in U.S. Pat. No. 7,446,190, and those described in International Patent Application Publication No.: WO 2014/055668.

In some embodiments, the antigen- or ligand-binding domain is an antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (V_{H}) and variable light (V_{L}) chains of a monoclonal antibody (mAb). The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)₂, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6^{th} ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352: 624-628 (1991).

Single-domain antibodies (sdAbs) are antibody fragments comprising all or a portion of the heavy chain variable domain or all or a portion of the light chain variable domain of an antibody. In certain embodiments, the single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragment is a scFv.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In some embodiments, the CAR comprises an antibody or an antigen-binding fragment (e.g., scFv) that specifically recognizes an antigen, such as an intact antigen, expressed on the surface of a cell.

In some embodiments, the CAR comprises a TCR-like antibody, such as an antibody or an antigen-binding fragment (e.g., scFv) that specifically recognizes an intracellular antigen, such as a tumor-associated antigen, presented on the cell surface as an MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR comprising an antibody or antigen-binding fragment that exhibits TCR-like specificity directed against peptide-MHC complexes also may be referred to as a TCR-like CAR.

In some embodiments, the recombinant receptors include recombinant T cell receptors (TCRs) and/or TCRs cloned from naturally occurring T cells. In some embodiments, a T cell receptor (TCR) comprises a variable α and β chains (also known as TCRα and TCRβ, respectively) or a variable γ and δ chains (also known as TCRγ and TCRδ, respectively), or a functional fragment thereof such that the molecule is capable of specifically binding to an antigen peptide bound to a MHC receptor. In some embodiments, the TCR is in the as form. Typically, TCRs that exist in αβ and γβ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to MHC molecules. In some embodiments, a TCR also can comprise a constant domain, a transmembrane domain and/or a short cytoplasmic tail (see, e.g., Janeway et al., Immunobiology: The Immune System in Health and Disease, 3^{rd} ed., Current Biology Publications, p. 4:33, 1997). For example, in some embodiments, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR for a target antigen (e.g., a cancer/tumor antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells, such as from a T cell (e.g., cytotoxic T cell), T cell hybridomas or other publicly available source. In some embodiments, the T-cells can be obtained from in vivo isolated cells. In some embodiments, a high-affinity T cell clone can be isolated from a patient and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15: 169-180 and Cohen et al. (2005) J Immunol. 175:5799-5808. In some embodiments, phage display is used to isolate TCRs against a target antigen (see, e.g., Varela-Rohena et al. (2008) Nat Med. 14: 1390-1395 and Li (2005) Nat Biotechnol. 23:349-354. In some embodiments, the TCR or antigen-binding portion thereof can be synthetically generated from knowledge of the sequence of the TCR of interest.

In addition to antibody fragments, non-antibody-based approaches have also been used to direct CAR specificity, usually taking advantage of natural ligand/receptor pairs that normally bind to each other. Cytokines, innate immune receptors, TNF receptors, growth factors, and structural proteins have all been successfully used as CAR antigen recognition domains (see, e.g., Chandran and Klebanoff, Immunol Rev. 2019 July; 290(1): 127-147).

In some embodiments, the receptor module (e.g., a CAR such as an antibody or antigen-binding fragment thereof), further includes a polypeptide spacer (or linker), which may be or include at least a portion of an immunoglobulin constant region or variant or modified version thereof, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the ligand-binding or antigen-recognition component, e.g., scFv, and the transmembrane domain. The spacer can be of a length that provides for increased responsiveness of the cell following antigen binding, as compared to in the absence of the spacer. Exemplary spacers include those having at least about 10 to 220 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. Exemplary spacers include IgG4 hinge alone, IgG4 hinge linked to CH2 and CH3 domains, or IgG4 hinge linked to the CH3 domain. The polypeptide spacer may also comprise a portion of the extracellular domain (preferably the membrane-proximal region) of receptors such as CD28 and CD8α. Exemplary spacers include, but are not limited to, those described in Hudecek et al. (2013) Clin. Cancer Res., 19:3153 or PCT patent publication number WO 2014/031687. In an embodiment, the hinge or spacer comprises the sequence of SEQ ID NO: 66.

In some embodiments, the antigen (or a ligand) recognized by the ligand-binding or antigen-recognition domain is a polypeptide. In some embodiments, the antigen (or a ligand) recognized by the ligand-binding or antigen-recognition domain is a carbohydrate or other molecule. In some embodiments, the antigen (or ligand) is selectively expressed or overexpressed on cells of the disease or condition, e.g., the tumor/cancer or pathogenic cells, relative to normal or non-targeted cells or tissues.

In some embodiments, the antigen (or a ligand) is a tumor antigen or cancer marker. In certain embodiments, the antigen is an integrin (e.g., αᵥβ₆ integrin, αᵥβ₃ integrin, integrin β₇), B cell maturation antigen (BCMA), B7-H6, carbonic anhydrase 9 (CA9, also known as CAIX or G250), CS1 (CD2 subset-1, CRACC, SLAMF7, and CD319), a cancer-testis antigen, cancer/testis antigen IB (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, fetal acetylcholine receptor, ganglioside GD2, O-acetylated GD2 (OGD2), ganglioside GD3, glycoprotein 100 (gp100), Her2/neu (receptor tyrosine kinase erbB2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, mesothelin, c-Met, murine cytomegalovirus (CMV), mucin 1 (MUC1), MUC 16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms Tumor 1 (WT-1), galectins (galectin-1, galectin-7), a pathogen-specific antigen, or an antigen associated with a universal tag, and/or biotinylated molecules, and/or molecules expressed by HIV, HCV, HBV or other pathogens such as bacteria and parasites. Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30. In an embodiment, the antigen targeted by the receptor is CD19. In a further embodiment, the receptor is an scFv comprising the sequence of SEQ ID NO:65. In an embodiment, the receptor is a receptor (e.g., scFv) present in an FDA-approved CAR T-cell therapy, e.g., the receptor of ABECMA® (idecabtagene vicleucel, anti-BCMA), BREYANZI® (lisocabtagene maraleucel, anti-CD19), TECARTUS™ (brexucabtagene autoleucel, anti-CD19), KYMRIAH™ (tisagenlecleucel, anti-CD19) or YESCARTA™ (axicabtagene ciloleucel, anti-CD19).

In an embodiment, the antigen is a protein expressed by pathogenic autoimmune cells, for example an autoantibody. In such cases, the ligand-binding or antigen-recognition domain is the antigen recognized by the autoantibody. An example of such antigen is the keratinocyte adhesion protein Dsg3, which could be useful to target B cells producing the autoantibodies to Dsg3 that cause the life-threatening autoimmune blistering disease Pemphigus vulgaris (PV). Another example is the muscle-specific kinase (MuSK), which could be useful to target B cells producing the autoantibodies to MuSK that cause autoimmune disease myasthenia gravis (MG).

In an embodiment, a plurality of recombinant receptors targeting a plurality of antigens are used. In a further embodiment, two recombinant receptors (i.e. two receptor modules) targeting two different antigens (e.g., CD19 and CD22, CD19 and CD20, BCMA and CS1) are used. In another embodiment, the ligand- or antigen-binding domain of the receptor module is bispecific, i.e. comprises a first domain that binds to a first ligand/antigen and a second domain that binds to a second ligand/antigen. This may be achieved, for example, by using two scFvs (binding to 2 different antigens) connected in tandem via a linker. In further embodiments, the ligand- or antigen-binding domain of the receptor module is multispecific, i.e. trispecific, tetraspecific, etc.

In an embodiment, the second synthetic transmembrane domain is fused the intracellular domain of a signaling protein, and more specifically of a signaling protein involved in the activation of an immune cell (e.g., T cell, B cell, NK cell). The activating intracellular domain, such as a T cell, B cell or NK cell activating domain, provides a primary and/or secondary activation signal. In some embodiments, the activating intracellular domain is an intracellular component of a TCR complex, such as a TCR CD3 chain that mediates T-cell activation and cytotoxicity, e.g., CD3 chain. In some embodiments, cell signaling modules include CD3 transmembrane domain, CD3 ζ intracellular signaling domains, and/or other CD transmembrane domains. Primary cytoplasmic signaling sequences that act in a stimulatory manner may comprise signaling motifs which are known as immunoreceptor tyrosine-based activation motifs or ITAMs. Examples of ITAM comprising primary cytoplasmic signaling sequences include those derived from TCR or CD3ζ, FcR gamma or FcR beta. In some embodiments, cytoplasmic signaling molecule(s) in the CAR comprise(s) a cytoplasmic signaling domain, portion thereof, or sequence derived from CD3ζ (e.g., SEQ ID NO: 69). In other embodiments, cytoplasmic signaling molecule(s) in the CAR comprise(s) a cytoplasmic signaling domain, portion thereof, or sequence derived from DAP10 or DAP12 (e.g., SEQ ID NO: 70).

In some embodiments, to promote full cell activation, a component for generating a secondary or co-stimulatory signal, for example the signaling domain of a costimulatory receptor such as CD28, 4-1BB, OX40, and ICOS, is also included in the modular receptor. In an embodiment, the intracellular domain for generating the secondary or co-stimulatory signal is attached to the first synthetic transmembrane domain, i.e. is part of the receptor module. In another embodiment, the intracellular domain for generating the secondary or co-stimulatory signal is attached to the second synthetic transmembrane domain, i.e. is part of the signaling module. The intracellular domains for generating the primary activation signal and the secondary or co-stimulatory signal may be included on the same signaling module, or may be incorporated into distinct signaling modules with a first signaling module comprising an intracellular domain for generating the primary activation signal and a second signaling module comprising an intracellular domain for generating the secondary or co-stimulatory activation signal. The skilled person would also understand that the modular receptor may include more than one intracellular domains for generating the primary and/or secondary activation signals. For example, a first intracellular domain for generating a first co-stimulatory signal may be included into the receptor module, and a second intracellular domain for generating a second co-stimulatory signal may be included into a signaling module. Alternatively, the two domains for generating the first and second co-stimulatory signals may be included (i) into the same receptor module (ii) into the same signaling module; or (iii) into two distinct receptor signaling modules.

The activating intracellular or cytoplasmic domain may be directly attached to the first and/or second synthetic transmembrane domain, or may be indirectly linked through a peptide linker. In some embodiments, a short oligo- or polypeptide linker comprising less than 100, 50, 40, 30, or 20 amino acids, for example a linker of between 2 and 20, 15 or 10 amino acids in length, such as one comprising glycines and/or serines, e.g., glycine-serine doublet, is present and forms a linkage between the transmembrane domain and the cytoplasmic signaling domain.

The present disclosure also provides one or more nucleic acids encoding the receptor module(s) and signaling module(s) defined herein. The nucleotide sequences encoding the receptor module(s) and signaling module(s) may be incorporated into the same nucleic acid molecules, or into distinct nucleic acid molecules.

In an embodiment, the one or more nucleic acids are comprised in a plasmid or a vector. Thus, the present disclosure also relates to a vector or plasmid comprising one or more nucleic acids encoding the receptor module(s) and signaling module(s) defined herein. The term “vector” is used to refer to a carrier into which a nucleic acid can be inserted for introduction into a cell where it can be replicated. The term “expression vector” or “nucleic acid vector” refers to a vector containing a nucleic acid or “expression cassette” coding for at least part of a gene product capable of being transcribed and “regulatory” or “control” sequences, which refer to nucleic acid sequences necessary for the transcription and possibly translation of an operably linked coding sequence in a particular host cell. In addition to control sequences that govern transcription and translation, expression vectors may contain nucleic acid sequences that serve other functions as well. The nucleic acids encoding the receptor module(s) and signaling module(s) may be under the transcriptional control of the same promoter/enhancer element (e.g., polycistronic), or of distinct promoter/enhancer elements.

In an embodiment, the vector further comprises a nucleic acid encoding a selectable marker or reporter protein. A selectable marker or reporter is defined herein to refer to a nucleic acid encoding a polypeptide that, when expressed, confers an identifiable characteristic (e.g., a detectable signal, resistance to a selective agent) to the cell permitting easy identification, isolation and/or selection of cells containing the selectable marker from cells without the selectable marker or reporter. Any selectable marker or reporter known to those of ordinary skill in the art is contemplated for inclusion as a selectable marker in the vector of the present disclosure. For example, the selectable marker may be a drug selection marker, an enzyme, or an immunologic marker. Examples of selectable markers or reporters include, but are not limited to, polypeptides conferring drug resistance (e.g., kanamycin/geneticin resistance), enzymes such as alkaline phosphatase and thymidine kinase, bioluminescent and fluorescent proteins such as luciferase, green fluorescent protein (GFP), yellow fluorescent protein (YFP), cyan fluorescent protein (CFP), blue fluorescent protein (BFP), citrine and red fluorescent protein from discosoma (dsRED), membrane bound proteins to which high affinity antibodies or ligands directed thereto exist or can be produced by conventional means, and fusion proteins comprising a membrane-bound protein appropriately fused to an antigen tag domain from, among others, hemagglutinin (HA) or Myc. The nucleic acid encoding the selectable marker or reporter protein may be under the control of the same promoter/enhancer as the one or more nucleic acids encoding the receptor module(s) and signaling module(s), or may be under the control of a distinct promoter/enhancer. In embodiments, the vector may comprise additional elements, such as one or more origins of replication sites (often termed “ori”), restriction endonuclease recognition sites (multiple cloning sites, MCS) and/or internal ribosome entry site (IRES) elements.

In an embodiment, the vector is a viral vector. The term “viral vector” as used herein refers to a recombinant virus capable of transducing cells and introducing their genetic material into the cells. In an embodiment, the viral vector is suitable for use in gene therapy applications. Examples of viral vectors that may be used in gene therapy include retroviruses (lentiviruses), adenoviruses, adeno-associated viruses (AAV), herpesviruses (herpes simplex viruses), alphaviruses, and vaccinia viruses (Poxviruses). In an embodiment, the viral vector is a lentiviral vector. As will be evident to one of skill in the art, the term “lentiviral vector” is used to refer to a lentiviral particle that mediates nucleic acid transfer. Lentiviral particles will typically include various viral components and sometimes also host cell components in addition to nucleic acid(s). In particular aspects, the terms “lentiviral vector,” “lentiviral expression vector” are used to refer to lentiviral transfer plasmids and/or infectious lentiviral particles.

In an embodiment, the lentiviral vector is a pseudotyped lentiviral vector. Pseudotyped lentiviral vectors consist of vector particles bearing enveloped proteins (glycoproteins, GP) derived from other enveloped viruses. Such particles possess the tropism of the virus from which the enveloped proteins are derived. One of the widely used glycoproteins for pseudotyping lentiviral vectors is the vesicular stomatitis virus GP (VSV-G), due to the very broad tropism and stability of the resulting pseudotypes. Pseudotyped lentiviral vectors are well known in the art, and several examples are described, for example, in Cronin et al., Curr. Gene Ther. 5(4):387-398. It includes lentiviral vectors pseudotyped with lyssavirus GPs, lymphocytic choriomeningitis virus (LCMV) GPs, alphavirus GPs (e.g., Ross River virus (RRV), Semliki Forest virus (SFV) and Sindbis virus GPs), Filovirus GPs (e.g., Marburg virus and Ebola Zaire virus GPs), gammaretrovirus GPs (e.g., ecotropic MLV, amphotropic 4070A MLV, 10A1 MLV, xenotropic NZB MLV, mink cell focus-forming virus, gibbon ape leukemia (GALV) virus, RD1 14 GPs), Vesicular Stomatitis Virus type-G (VSV-G), Measles-Virus Lentiviral vector (MV-LV), Baboon envelop (BaEV)-LVs and baculovirus GPs (GP64).

In an embodiment, the vector is an episomally-maintained viral vector or non-integrating vector, such as a Sendai virus or vector. Such vectors are not integrated into the genome, but are maintained episomally with cell division due to scaffold/matrix attachment region presence inside vector (see, e.g., Giannakopoulos A et al., J Mol Biol. 2009 Apr. 17; 387(5):1239-49; and Haase et al., BMC Biotechnol. 2010; 10: 20).

In another embodiment, the vector is a non-viral vector, for example nude DNA, a liposome, a polymerizer or a molecular conjugate.

In another aspect, the present disclosure provides a cell (host cell, engineered cell) comprising the above-mentioned nucleic acids or vector/plasmid so as to express the modular chimeric receptor defined herein. In an embodiment, the cell is a primary cell, for example a brain/neuronal cell, a peripheral blood cell (e.g., a B or T lymphocyte, a monocyte, a NK cell), a cord blood cell, a bone marrow cell, a cardiac cell, an endothelial cell, an epidermal cell, an epithelial cell, a fibroblast, hepatic cell or a lung/pulmonary cell. In an embodiment, the cell is a bone marrow cell, peripheral blood cell or cord blood cell. In a further embodiment, the cell is an immune cell, such as a T cell (e.g., a CD8⁺ T cell), a B cell or a NK cell. In another embodiment, the immune cell is a regulatory T cell (T_{reg}).

In an embodiment, the cell is a stem cell. The term “stem cell” as used herein refers to a cell that has pluripotency which allows it to differentiate into a functional mature cell. It includes primitive hematopoietic cells, progenitor cells, as well as adult stem cells that are undifferentiated cells found in various tissue within the human body, which can renew themselves and give rise to specialized cell types and tissue from which the cells came (e.g., muscle stem cells, skin stem cells, brain or neural stem cells, mesenchymal stem cell, lung stem cells, liver stem cells). In an embodiment, the cell is a mammalian cell, for example a human cell.

In another aspect, the present disclosure provides a composition comprising the cell described herein. The composition may comprise one or more carrier or excipient, e.g. a buffer, a saline solution, a preservative, etc. In an embodiment, the composition is a pharmaceutical composition comprising at least one pharmaceutically acceptable carrier or excipient. An “excipient,” as used herein, has its normal meaning in the art and is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art (see, e.g., Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). Pharmaceutical compositions may be prepared using standard methods known in the art by mixing the cells with one or more optional pharmaceutically acceptable carriers, excipients and/or stabilizers. The excipient may be selected for administration of the composition by any routes, for example, for intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration. In an embodiment, the pharmaceutical composition is formulated for injection, e.g. as a solution, suspension, or emulsion, including localized injection, catheter administration, systemic injection, intravenous injection, intraperitoneal injection, subcutaneous injection or parenteral administration.

Pharmaceutical compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium comprising, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can also be lyophilized. The compositions can comprise auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers comprising the antibody, which matrices are in the form of shaped articles, e.g. films, or microcapsules.

The formulations to be used for in vivo administration are generally sterile. Sterility may be readily accomplished, e.g., by filtration through sterile filtration membranes.

The present disclosure also relates to a method for treating a disease, condition or disorder in a subject, the method comprising administering a cell expressing the modular chimeric receptor described herein. The present disclosure also relates to the use of a cell expressing the modular chimeric receptor described herein for treating a disease, condition or disorder in a subject. The present disclosure also relates to the use of a cell expressing the modular chimeric receptor described herein for the manufacture of a medicament for treating a disease, condition or disorder in a subject.

The disease or condition that may treated can be any in which expression of an antigen, or of a cell expressing the antigen (e.g., a tumor cell expressing a tumor antigen), is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g., cancer), autoimmune or inflammatory disease (e.g., arthritis, rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant), or an infectious disease, e.g. caused by a bacterial, viral or other pathogen. In particular embodiments, the modular chimeric receptor, e.g., the CAR, specifically binds to the antigen associated with the disease or condition or expressed by a cell associated with the disease or condition. In an embodiment, the disease, condition or disorder is cancer or an infectious disease, and the modular receptor comprises a ligand-binding domain that recognizes an antigen expressed by the tumor cell or infected cell.

The tumor may be a solid tumor or a hematologic (blood) tumor.

In an embodiment, the cancer is heart sarcoma, lung cancer, small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma (e.g., Ewing's sarcoma, Karposi's sarcoma), lymphoma, chondromatous hamartoma, mesothelioma; cancer of the gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); cancer of the genitourinary tract, for example, kidney cancer (adenocarcinoma, Wilm's tumor [nephroblastoma], lymphoma, leukemia), bladder and/or urethra cancer (squamous cell carcinoma, transitional cell carcinoma, adenocarcinoma), prostate cancer (adenocarcinoma, sarcoma), testis cancer (seminoma, teratoma, embryonal carcinoma, teratocarcinoma, choriocarcinoma, sarcoma, interstitial cell carcinoma, fibroma, fibroadenoma, adenomatoid tumors, lipoma); liver cancer, for example, hepatoma (hepatocellular carcinoma, HCC), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone cancer, for example, osteogenic sarcoma (osteosarcoma), fibrosarcoma, malignant fibrous histiocytoma, chondrosarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; cancer of the nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); cancer of the reproductive system, for example, gynecological cancer, uterine cancer (endometrial carcinoma), cervical cancer (cervical carcinoma, pre-tumor cervical dysplasia), ovarian cancer (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulvar cancer (squamous cell carcinoma, intraepithelial carcinoma, adenocarcinoma, fibrosarcoma, melanoma), vaginal cancer (clear cell carcinoma, squamous cell carcinoma, botryoid sarcoma (embryonal rhabdomyosarcoma), fallopian tube cancer (carcinoma); placenta cancer, penile cancer, prostate cancer, testicular cancer; cancer of the hematologic system, for example, blood cancer (acute myeloid leukemia (AML), chronic myeloid leukemia (CML), acute lymphoblastic leukemia (ALL), chronic lymphocytic leukemia (CLL), myeloproliferative diseases, multiple myeloma, myelodysplastic syndrome), Hodgkin's disease, non-Hodgkin's lymphoma [malignant lymphoma]; cancer of the oral cavity, for example, lip cancer, tongue cancer, gum cancer, palate cancer, oropharynx cancer, nasopharynx cancer, sinus cancer; skin cancer, for example, malignant melanoma, cutaneous melanoma, basal cell carcinoma, squamous cell carcinoma, Karposi's sarcoma, moles dysplastic nevi, lipoma, angioma, dermatofibroma, and keloids; adrenal gland cancer: neuroblastoma; and cancers of other tissues including connective and soft tissue, retroperitoneum and peritoneum, eye cancer, intraocular melanoma, and adnexa, breast cancer (e.g., ductal breast cancer), head or/and neck cancer (head and neck squamous cell carcinoma), anal cancer, thyroid cancer, parathyroid cancer; secondary and unspecified malignant neoplasm of lymph nodes, secondary malignant neoplasm of respiratory and digestive systems and secondary malignant neoplasm of other sites.

In a further embodiment, the cancer is a hematologic cancer, such as a lymphoma, a leukemias, and/or a myeloma (e.g., B-cell, T-cell, and myeloid leukemias, lymphomas, and multiple myelomas), for example acute lymphoblastic leukemia (ALL) (e.g., relapsed/refractory B-cell precursor ALL), diffuse large B-cell lymphoma (DLBCL), Hodgkin's lymphoma (e.g., refractory Hodgkin's lymphoma), acute myeloid leukemia (AML), and multiple myeloma. In an embodiment, the cancer is refractory to standard chemotherapy and/or is a relapsing cancer.

The infectious disease may be a disease caused by any pathogenic infection, such as a viral, bacterial, parasitic (e.g., protozoal) or fungal infection, for example human immunodeficiency virus (HIV) or cytomegalovirus (CMV) infection.

The cells (engineered cells expressing the modular chimeric receptor) or compositions comprising same may administered to a subject or patient having the particular disease or condition to be treated, e.g., via adoptive cell therapy such as adoptive T cell therapy, or stem cell therapy. Methods for administration of engineered cells for adoptive cell therapy are known and may be used in connection with the provided methods and compositions. For example, adoptive T cell therapy methods are described, e.g., in U.S. Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease (e.g., reduction of tumor volume, reduction of viral/bacterial load), preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

In some embodiments, the cell therapy, e.g., adoptive cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or super type as the first subject. The cells can be administered by any suitable means. Dosing and administration may depend in part on whether the administration is brief or chronic. Various dosing schedules include but are not limited to single or multiple administrations over various time-points, bolus administration, and pulse infusion.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents.

The one or more additional active agents or therapies may be one or more chemotherapeutic agents, immunotherapies, checkpoint inhibitors, cell-based therapies, etc. Examples of chemotherapeutic agents suitable for use in combination with the cells described herein include, but are not limited to, vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, retinoids (such as all-trans retinoic acids or a derivatives thereof); geldanamycin or a derivative thereof (such as 17-AAG), and other cancer therapeutic agents recognized in the art. In some embodiments, chemotherapeutic agents for use in combination with the cells described herein comprise one or more of adriamycin, colchicine, cyclophosphamide, actinomycin, bleomycin, duanorubicin, doxorubicin, epirubicin, mitomycin, methotrexate, mitoxantrone, fluorouracil, carboplatin, carmustine (BCNU), methyl-CCNU, cisplatin, etoposide, interferons, camptothecin and derivatives thereof, phenesterine, taxanes and derivatives thereof (e.g., taxol, paclitaxel and derivatives thereof, taxotere and derivatives thereof, and the like), topetecan, vinblastine, vincristine, tamoxifen, piposulfan, nab-5404, nab-5800, nab-5801, Irinotecan, HKP, Ortataxel, gemcitabine, Oxaliplatin, Herceptin®, vinorelbine, Doxil®, capecitabine, Alimta®, Avastin®, Velcade®, Tarceva®, Neulasta®, lapatinib, sorafenib, erlotinib, erbitux, derivatives thereof, and the like. In some embodiments, the one or more additional agents include one or more cytokines, such as IL-2, for example, to enhance persistence. In some embodiments, the methods comprise administration of a chemotherapeutic agent.

### MODE(S) FOR CARRYING OUT THE INVENTION

The present invention is illustrated in further details by the following non-limiting examples.

### Example 1: Design of Modular Membrane Receptors

Modular membrane receptors capable of assembling in a way that would not interfere or compete with endogenous receptors were developed by performing a knowledge-based mutational screen within the TM domains of the ligand-binding (Rc) and signaling (Sig.) modules of the representative immune receptors NKG2C and KIR2DS3 (KI2S3) and the representative signalling module DAP12 (FIGS. 1A-D). The receptor module was fused to an scFv targeting CD19 to create a modular antigen receptor (FIG. 1C). This facilitates the measurement of surface expression, signalling potential and cell activation. A library of KIR2DS3 Rc (mCherry) and Sig. (YFP) mutants was created (FIG. 1B), while the NKG2C Rc were expressed with DAP12 Sig from a bicistronic expression vector separated by a 2A peptide (FIG. 1A). The assembly of endogenous modular receptors require the proper positioning an interaction of charged amino acids in the Rc (positively charged residue: lysine, arginine) and Sig modules (negatively charged residue: aspartate, glutamate). By repositioning the charged amino acids within the TM domains, the selectivity of assembly can be achieved by matched, but not all, positionally shifted Rc and Sig modules. This can pre-empt the interaction of endogenous (WT) modules with shifted (−4, +4 etc.) modules (FIG. 1D). Table I shows the sequences of the TM domains of Rc and Sig tested in the studies described herein, and Table 11 shows the sequences of the various modules tested in the studies described herein.

TABLE I
Sequences of the TM domains of Rc and Sig tested in the studies described herein

| | Amino acid sequence Orientation | SEQ ID |
|---|---|---|
| Rc.Mod. TMs | (Extra-cell.........................Intra-cell) | NO: |
| KIR2DS3 | N-term...............................C-term | |
| WT | VLIGTSVVKLPFTILLFFLLH | 73 |
| RS1 | VLIGTSVVLLPFKILLFFLL | 53 |
| RS2 | VLIGKSVVLLPFTILLFFLL | 54 |
| RS3 | VLILLLLLKLLLLLLLFFLL | 55 |
| RS4 | VLILLLLLLLLLKLLLFFLL | 56 |
| RS5 | VLILKLLLLLLLLLLLFFLL | 57 |
| R$1.2 | VLIILLVGTSVVKLLLFFLL | 58 |
| RS1.3 | VLIGTSVVTLPFKILLFFLL | 59 |
| RS4.2 | VLILLLLLKLLLKLLLFFLL | 60 |
| RS4.3 | VLILLLLGKLLLKLLLFFLL | 61 |
| RS4.4 | VLILLLLGLLLLKLLLFFLL | 62 |
| RS4.5 | VLILLLLLALLLKLLLFFLL | 63 |
| RS4.6 | VLILLLLLTLLLKLLLFFLL | 64 |
| NKG2C | C-term...............................N-term | |
| WT | ELFPILVITKLVTAMLVISIIGLV | 46 |
| (+4) | ELFPILVITALVTKMLVISIIGLV | 48 |
| (-4) | ELFPIKVITLLVTAMLVISIIGLV | 47 |
| Sig. Mod. TMs | | |
| DAP12 | N-term...............................C-term | |
| WT | GVLAGIVMGDLVLTVLIALAVYFL | 42 |
| DS1 | GVLAGIVMGALVLDVLITLAVYFL | 44 |
| DS2 | GVLALAVLGIVMGDVLITLAVYFL | 45 |
| DS3 | GVLAGDVMGTLVLIVLIALAVYFL | 43 |

TABLE II
Amino acid sequences of the various modules tested in the studies described
herein.

| Name | Amino acid sequence | SEQ ID NO: |
|---|---|---|
| mCAR_CD19 | MALPVTALLL PLALLLHAAR PDIQMTQTTS SLSASLGDRV | 65 |
| scFv | TISCRASQDI SKYLNWYQQK PDGTVKLLIY HTSRLHSGVP | |
| | SRFSGSGSGT DYSLTISNLE QEDIATYFCQ QGNTLPYTFG | |
| | GGTKLEITGG GGSGGGGSGG GGSEVKLQES GPGLVAPSQS | |
| | LSVTCTVSGV SLPDYGVSWI RQPPRKGLEW LGVIWGSETT | |
| | YYNSALKSRL TIIKDNSKSQ VFLKMNSLQT DDTAIYYCAK | |
| | HYYYGGSYAM DYWGQGTSVT VSS | |
| mCAR_CD8 | TTTPAPRPPT PAPTIASQPL SLRPEACRPA AGGAVHTRGL | 66 |
| alpha hinge | DFAC | |
| mCAR_CD8 | HLH | — |
| alpha hinge | | |
| spacer | | |
| mCAR_ | RRKPSLLAHP GRLVKSEETV ILQCWSDVMF EHFLLHREGT | 67 |
| KIR2DS2EC | FNDTLRLIGE HIDGVSKANF SIGRMRQDLA GTYRCYGSVP | |
| | HSPYQFSAPS DPLDIVITGL YEKPSLSAQP GPTVLAGESV | |
| | TLSCSSWSSY DMYHLSTEGE AHERRFSAGP KVNGTFQADF | |
| | PLGPATQGGT YRCFGSFHDS PYEWSKSSDP LLVSVTGNPS | |
| | NSWPSPTEPS SKTGNPRHLH | |
| mCAR_CD28 | HRSKRSRLLH SDYMNMTPRR PGPTRKHYQP YAPPRDFAAY | 68 |
| cyto domain | RS | |
| mCAR_CD3 | RVKFSRSADA PAYQQGQNQL YNELNLGRRE EYDVLDKRRG | 69 |
| zeta | RDPEMGGKPQ RRKNPQEGLY NELQKDKMAE AYSEIGMKGE | |
| cyto domain | RRRGKGHDGL YQGLSTATKD TYDALHMQAL PPR | |
| mCAR_DAP12 | GRLVPRGRGA AEAATRKQRI TETESPYQEL QGQRSDVYSD | 70 |
| cyto domain | LNTQRPYYK | |
| SOC- | RSKRSRLLHS DYMNMTPRRP GPTRKHYQPY APPRDFAAYR | 71 |
| CAR_CD28/CD3 | SSGLRPVQAQ AQSDCSCSTV SPGVLAGSRL AVYFLGRVKF | |
| zeta cyto | SRSADAPAYQ QGQNQLYNEL NLGRREEYDV LDKRRGRDPE | |
| domain | MGGKPQRRKN PQEGLYNELQ KDKMAEAYSE IGMKGERRRG | |
| | KGHDGLYQGL STATKDTYDA LHMQALPPR | |
| SOC- | MALPVTALLL PLALLLHAAR PDIQMTQTTS SLSASLGDRV | 72 |
| CAR_CD19- | TISCRASQDI SKYLNWYQQK PDGTVKLLIY HTSRLHSGVP | |
| CD8hinge and | SRFSGSGSGT DYSLTISNLE QEDIATYFCQ QGNTLPYTFG | |
| TM | GGTKLEITGG GGSGGGGSGG GGSEVKLQES GPGLVAPSQS | |
| | LSVTCTVSGV SLPDYGVSWI RQPPRKGLEW LGVIWGSETT | |
| | YYNSALKSRL TIIKDNSKSQ VFLKMNSLQT DDTAIYYCAK | |
| | HYYYGGSYAM DYWGQGTSVT VSSTTTPAPR PPTPAPTIAS | |
| | QPLSLRPEAC RPAAGGAVHT RGLDFACDIY IWAPLAGTCG | |
| | VLLLSLVITL Y | |

### Example 2: Identification of Synthetic Signaling Modules that do not Assemble with the Native KIRD2S Rc Module

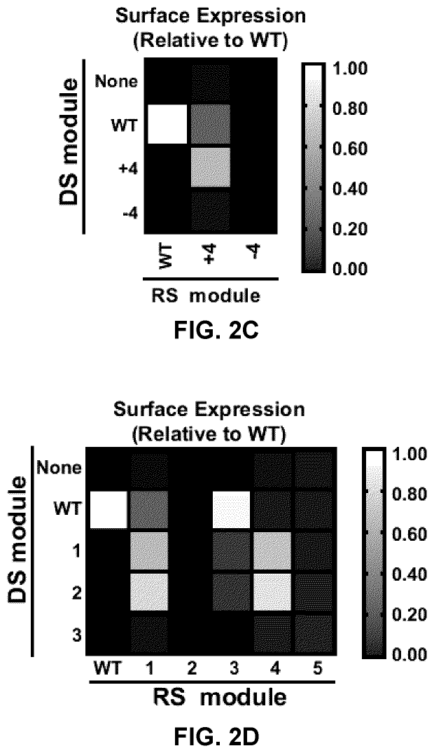

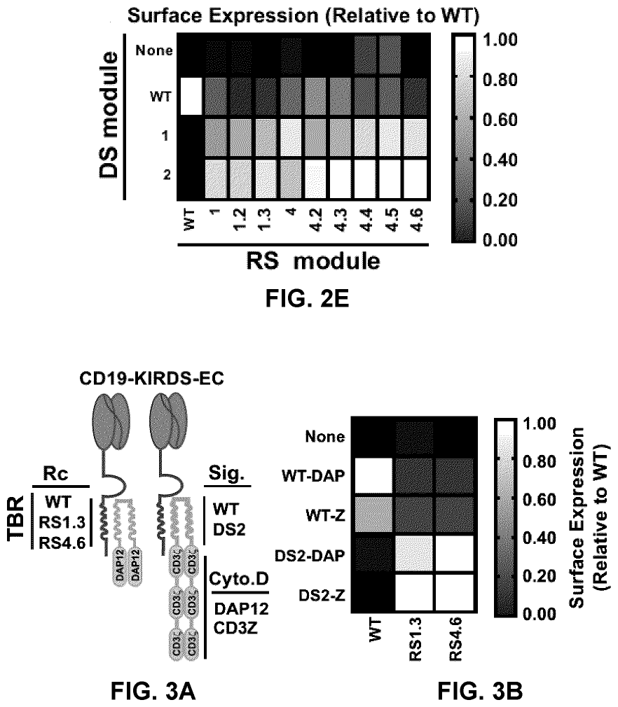

As modular immune receptors require assembly for surface expression, the surface expression of each Rc/Sig combinations was compared to that of the native NKG2C (FIG. 2A) and KIR2DS/DAP12 (FIGS. 2B-E) for efficiency and specificity of assembly. Using this approach, synthetic signaling modules that did not assemble with the native (WT) KIRD2S Rc module (FIGS. 2C-E, black boxes), as well as synthetic receptor (RS) modules that required assembly for proper surface expression and capable of assembling with various OS modules (FIGS. 2C-E, white and grey boxes), were successfully identified. The approach was shown to work for the transmembrane domain of a Type I (KIR2DS3, extracellular N-terminal) or type 11 (NKG2C, intracellular N-terminal) receptor, demonstrating that the approach does not depend on the orientation of the receptor. Also, replacing the cytoplasmic domain of DAP12 by that of CD3zeta in the signaling module gave similar results, providing evidence that the approach does not depend on the nature of the cytoplasmic signaling domain.

### Example 3: Testing of the Approach Using Another Intracellular Domain

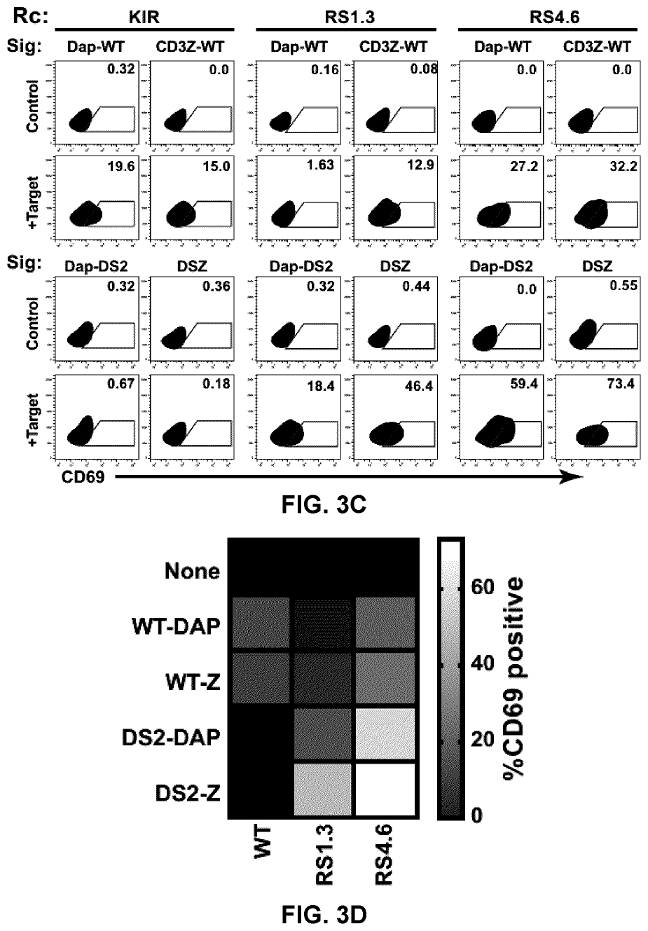

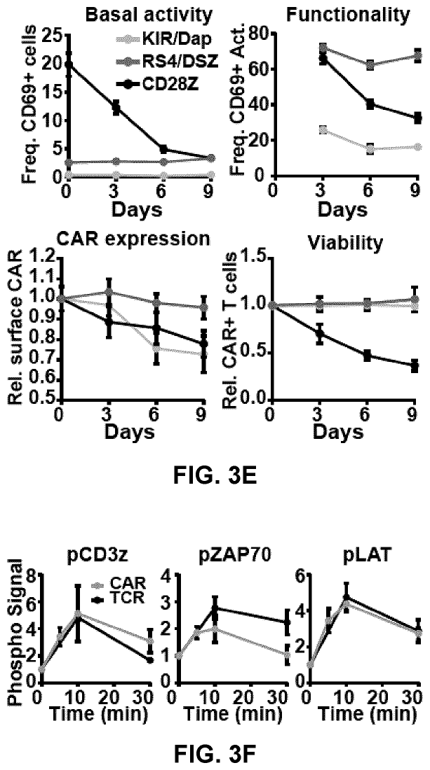

It was next tested whether the approach was suitable regardless of the intracellular domain of the Signalling module. After additional TM domain optimization of the RS1 and RS4 synthetic TM to obtain the RS1.3 and RS4.6 scaffolds, their assembly and surface expression was assessed in a system in which the normal DAP12 cytoplasmic domain was replaced with the CD3ζ signalling domain (FIGS. 3A-B). The RS4.6 scaffold retained a preference for assembly and surface expression with the DS2 module, with greater surface expression when assembled with the DS2 module containing the CD3ζ signaling cue (DSZ) (FIG. 3B). Next, the activation capabilities of these novel mCARs were compared to that of the native KIR/DAP12 receptor by incubating the CD19-mCAR expressing Jurkat cell lines with CD19-positive patient-derived lymphoma the cell line HT, and monitoring CD69 expression 16 hours post-incubation (FIG. 3C). The results depicted in FIG. 3D clearly illustrate the superiority of the RS4.6/DSZ mCAR for its ability to activate T cells in a target cell-specific context. mCAR expression and functional stability was assessed following continuous culture and repeated stimulation. In these assays, the KIR/DAP12 and CD28ζ CARs were also included for comparison purposes. First, the basal activity status of the CD28ζ CAR could be observed in the presence of CD69+ cells in the absence of target cells, which lead to reduced cell viability, and functionality, which was either caused by exhaustion and/or natural selection of cells displaying reduced surface expression (FIG. 3E, black line). Contrastingly, the mCAR developed herein (RS4.6/DSZ) maintained functionality, expression and viability (FIG. 3E, dark grey). Moreover, the signaling kinetics of this modular CAR, when triggered by target cells, mimics those observed for immune receptors (FIG. 3F).

### Example 4: Cytotoxic Activity of the mCARs

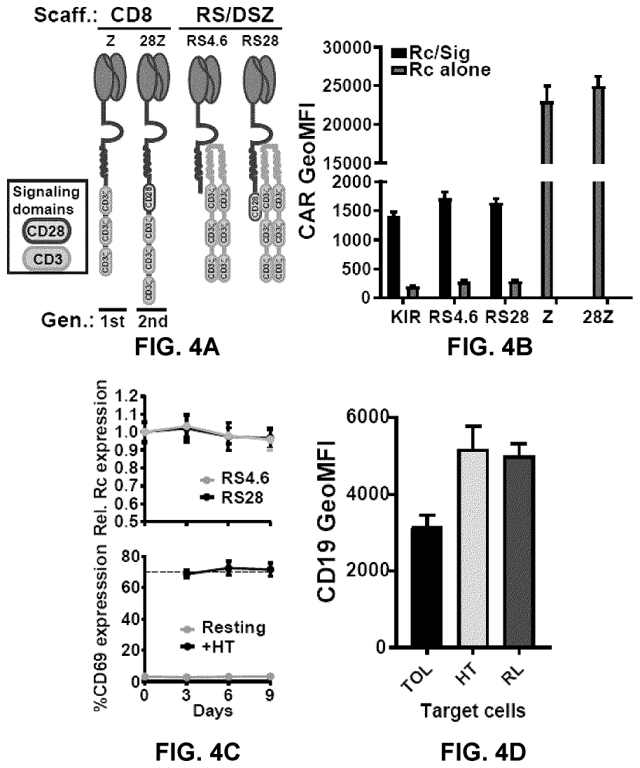

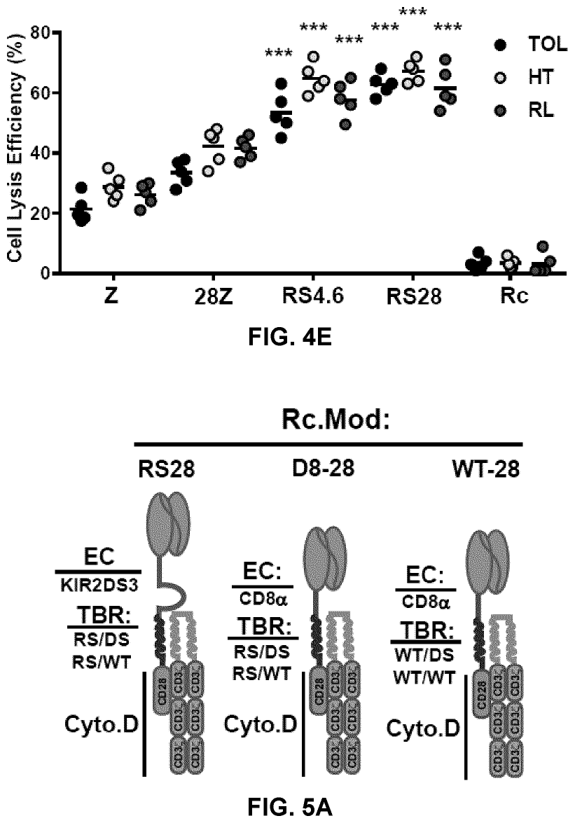

The cytolytic ability of the modular CARs was validated in primary human CD8 T cells, and compared to that of the CD19-specific CD3ζ and CD28ζ stemming from 1^{st} and 2^{nd} generation CARs. An additional mCAR was included in this assay to recapitulate the added activity of CD28 by fusing its cytoplasmic domain to the Rc module (FIG. 4A). After quantifying surface expression levels (FIG. 4B), stability and activating functionality (FIG. 4C) of all these receptors, the cytolytic assay was performed (FIG. 4E). To do so, GFP-expressing patient-derived cell lines Toledo (TOL), HT and RL that express varying amounts of surface CD19 were incubated with CAR-transduced CD8 T cells for 33-36 hours (FIG. 4D). GFP leaching into the culture media was then monitored by quantifying fluorescence in the supernatant using a plate reader. Results clearly illustrate that the CD19-mCARs developed herein exhibit superior cytolytic capabilities and demonstrate added safety, as evidenced by the fact that the expression of the Rc module alone is insufficient for cell activation and function (FIG. 4E).

### Example 5: Assembly and Functions of Modular CARs Based on a CD8alpha Scaffold

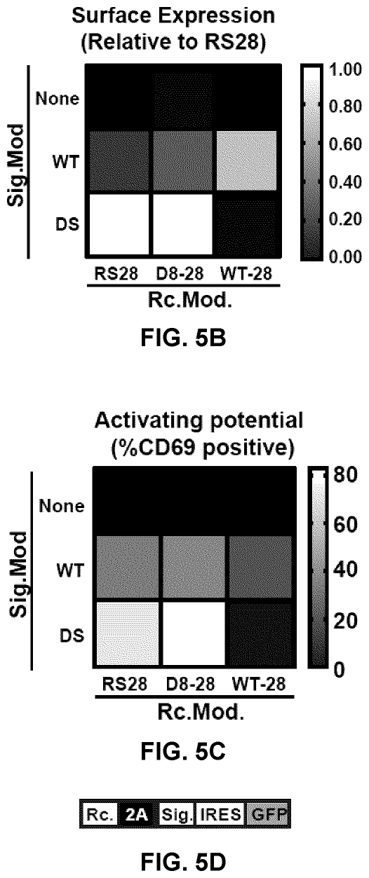

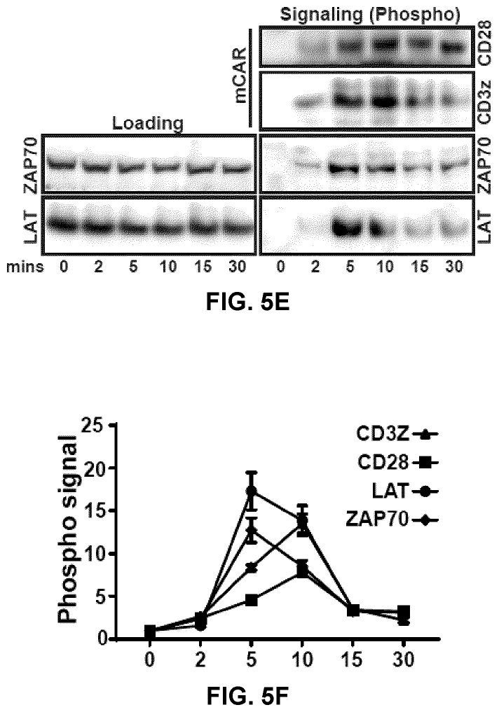

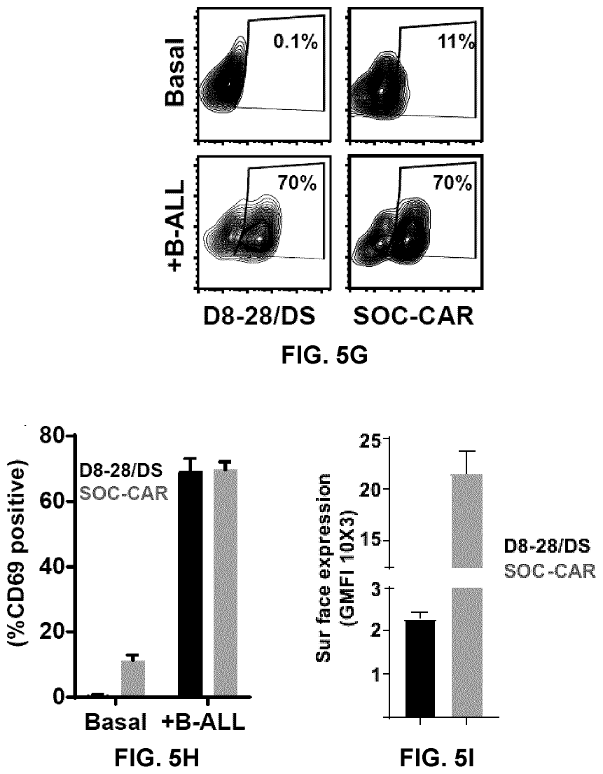

In order to determine whether mCAR assembly through TBR coordination was specific to the original KIR2DS3 extracellular (EC) scaffold from which the TBR was designed from, it was replaced with that of CD8a hinge region commonly used in Standard-Of-Care CAR (SOC-CAR), and is present in the SOC-CAR used in these assays (FIG. 5A). To determine the specificity and efficacy of surface expression as well as activation potential of the novel mCARs built with the CD8alpha (CD8α) hinge region, they were benchmarked to the best-in-class RS28 Rc Module. The D8-28 Rc module contains the CD19scFv, the CD8a hinge region, the HLH spacer peptide followed by the RS4.6 TBR and CD28 cytoplasmic domain. The WT-28 module contains the CD19scFv, the CD8a hinge region, the HLH spacer peptide followed by the WT TBR. To determine specificity and necessity of association for surface expression, cells were co-transduced the Sig modules containing either the DS2 (DS) or WT TBRs as well as the CD3zeta cytoplasmic domains (FIG. 5A). Cells were co-transduced to express each component in every combination, as well as Rc module only (Sig.Mod. None) in order to assess leakiness in the absence of assembly. As previously done, mCAR surface expression was determined using fluorescent tetramerized CD19 and FACS analysis. The D8-28 Rc modules retained its necessity of assembly for surface expression as well as a preference for assembly with TBR-matched Sig module (FIG. 5B). As observed for the WT TBR containing KIR2DS3 scaffold, the WT-28 Rc module failed to assemble with the DS2 TBR-containing Sig module and to be expressed at the surface (FIG. 5B).

Next, the activating potential of the new scaffold-containing Rc modules was assessed by co-culturing the mCAR-expressing Jurkat cells with CD19-positive B-ALL leukemic cells for 16 hrs prior to evaluating surface expression of CD69 as described previously. Interestingly, the D8-28/DS mCAR provided higher activating potential than the RS28/DS and WT-28 mCAR (FIG. 5C). In view of its superior activating potential, the D8-28/DS mCAR was further characterized. First, the Rc and Sig modules were recloned into a polycistronic cassette enabling the production of two separate proteins following the cleavage of the P2A peptide (FIG. 5D). Of note, the new CD19-D8-28/DS mCAR has a reduced cargo size of roughly 600 bp when compared to the RS28/DS mCAR. To investigate the signaling capabilities of the novel D8-28/DS mCAR, mCAR expressing Jurkat cells were co-incubated with CD19-positive B-ALL leukemic cells (HT cells) and activated by performing a pulse spin, forcing the cells to contact each other. Following various incubation periods, cells were harvested and prepared for Western blot analysis. To determine signaling kinetics, the phosphorylated CD3 zeta, CD28, ZAP70 and LAT were probed using phospho-specific antibodies. To normalize phospho signals to protein abundance, total ZAP70 and LAT were also probed (FIG. 50). As observed for the RS28/DS mCAR, the D8-28/DS mCAR robustly became phosphorylated (phospho CD3z and CD28) following engagement with CD19-positive target cells and initiated rapid signal transduction (phospho ZAP70 and LAT). As observed previously mCAR and downstream signaling components were then rapidly dephosphorylated in a fashion similar to that observed following TCR engagement (FIG. 5F, and reference FIG. 3F).

Finally, the activating potential as well as basal signaling of the novel D8-28/DS mCAR were benchmarked to those of the 28Z SOC-CAR (FIG. 4A) by incubating the CAR-expressing cells, or not, with CD19-positive B-ALL leukemic cells and probing for CD69 surface expression. The D8-28/DS cells showed no basal mCAR activity in the absence of target cells (0.1% CD69+), but became robustly activated in their presence (70% CD69+), indicating the necessity of target cells for mCAR-T activation (FIGS. 5G-H). Contrastingly, the 28Z-SOC-CAR expressing showed significant basal activation in the absence of target cells at day 6 post-transduction (11% CD69+), but also reached robust activation in their presence (70% CD69+). These results highlight the superior safety profile of the D8-28/DS relative to SOC-CARs because of their lack of tonic signaling. Considering the reduced surface expression of the D8-28/DS mCAR relative to the 28Z SOC-CAR, these results also confirm their superiority in potency (FIG. 5I).

### Example 6: Modular CARs Enable the Formation of Stable Immune Synapses and Promote Rapid Tumor Cell Killing In Vitro

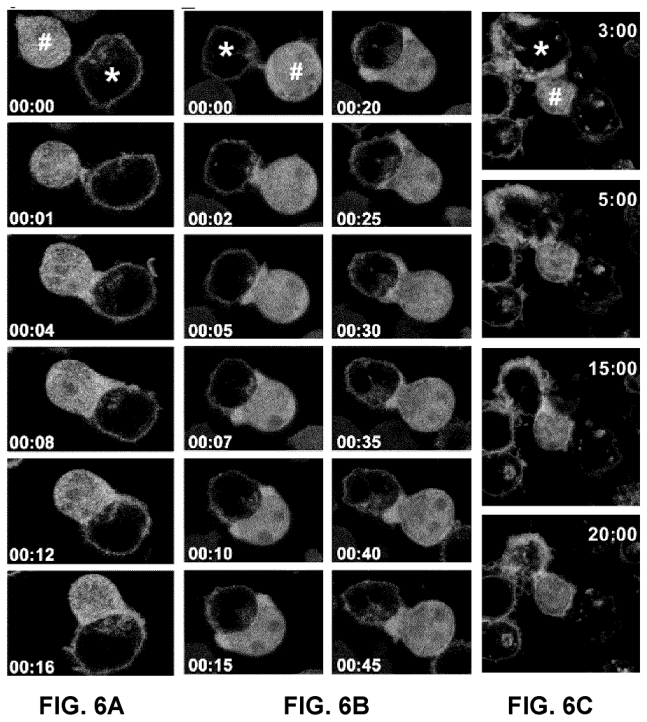

One aspect that may be at cause for the complications observed for patients treated with SOC-CARs is the inability of these receptors to engage in stable immunological synapse formation, but rather engage the formation of less-stable immunological kinapses. To test whether the new D8-28/DS mCAR enable the formation of stable immune synapse, an in vitro imaging assay was performed. Primary human T cells were transduced to express the D8-28/DS mCAR as a P2A polycistronic expression cassette and then sorted based on fluorescence provided by the GFP as well as CD4 or CD8 phenotype. The mCAR-expressing T cells were then co-incubated with a CD129-positive B-ALL leukemic cells line expressing the PM-targeted and live cell indicator Lact-C2-RFP. Lact-C2-RFP binds to phosphatidylserine (PS) within the inner leaflet of the plasma membrane at steady-state. When cells die, massive influx of calcium activate scramblase such as TMEM16F and inhibit APLTs, which causes a redistribution of PS to the outer leaflet of the PM and disengages Lact-C2-RFP from the membrane towards vesicular organelles. To perform the assay, Lact-C2-RFP expressing B-ALL cells were placed in a flow cell are were allowed to adhere for 5 mins. After which, CD4 or CD8 T cells that express the CD19-mCAR were injected into the flow cell and imaging commenced. Images for GFP and RFP were acquired over the course of 45 minutes, at 15 seconds interval. CD4 mCAR-T cells rapidly engaged CD19-positive leukemic cells and maintained intimate cell contact, i.e. immunological synapse, for the duration of the imaging (FIG. 6A). This is similar to what is observed for TCR-engaged T cells with cognate peptide-presenting antigen presenting cells (APCs). Similar to CD4 mCAR-T cells, the CD8 mCAR-T cells also rapidly engaged target cells and maintained cell/target contact for long periods of time, although the immune synapses were more dynamic in nature (FIG. 6C). Importantly, however, the CD8 mCAR-T cell remained in close contact with a single target cell until it killed it, making this very different to what had been observed for SOC-CAR, which display multiple engagement and disengagement with multiple cells before target cells death, which also required contacts from multiple CD8 CAR-T cells.

Although the present technology has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Zhang, T. et al. Efficiency of CD19 chimeric antigen receptor-modified T cells for treatment of B cell malignancies in phase I clinical trials: a meta-analysis. Oncotarget 6, 33961-33971, doi:10.18632/oncotarget.5582 (2015).
2. Dotti, G., Savoldo, B. & Brenner, M. Fifteen years of gene therapy based on chimeric antigen receptors: “are we nearly there yet?”. Hum Gene Ther 20, 1229-1239, doi:10.1089/hum.2009.142 (2009).
3. Milone, M. C. et al. Chimeric receptors containing CD137 signal transduction domains mediate enhanced survival of T cells and increased antileukemic efficacy in vivo. Mol Ther 17, 1453-1464, doi:10.1038/mt.2009.83 (2009).
4. Shen, C. J. et al. Chimeric antigen receptor containing ICOS signaling domain mediates specific and efficient antitumor effect of T cells against EGFRvIII expressing glioma. J Hematol Oncol 6, 33, doi:10.1186/1756-8722-6-33 (2013).
5. Tang, X. Y. et al. Third-generation CD28/4-1BB chimeric antigen receptor T cells for chemotherapy relapsed or refractory acute lymphoblastic leukaemia: a non-randomised, open-label phase I trial protocol. BMJ Open 6, e013904, doi:10.1136/bmjopen-2016-013904 (2016).
6. Wei, G., Ding, L., Wang, J., Hu, Y. & Huang, H. Advances of CD19-directed chimeric antigen receptor-modified T cells in refractory/relapsed acute lymphoblastic leukemia. Exp Hematol Oncol 6, 10, doi:10.1186/s40164-017-0070-9 (2017).
7. Abate-Daga, D. & Davila, M. L. CAR models: next-generation CAR modifications for enhanced T-cell function. Mol Ther Oncolytics 3, 16014, doi:10.1038/mto.2016.14 (2016).
8. Sadelain, M., Brentjens, R. & Riviere, I. The basic principles of chimeric antigen receptor design. Cancer Discov 3, 388-398, doi:10.1158/2159-8290.CD-12-0548 (2013).
9. Salter, A. I. et al. Phosphoproteomic analysis of chimeric antigen receptor signaling reveals kinetic and quantitative differences that affect cell function. Sci Signal 11, doi:10.1126/scisignal.aat6753 (2018).
10. Call, M. E., Pyrdol, J., Wiedmann, M. & Wucherpfennig, K. W. The organizing principle in the formation of the T cell receptor-CD3 complex. Cell 111, 967-979 (2002).
11. Call, M. E., Pyrdol, J. & Wucherpfennig, K. W. Stoichiometry of the T-cell receptor-CD3 complex and key intermediates assembled in the endoplasmic reticulum. EMBO J 23, 2348-2357, doi:10.1038/sj.emboj.7600245 (2004).
12. Call, M. E. & Wucherpfennig, K. W. Molecular mechanisms for the assembly of the T cell receptor-CD3 complex. Mol Immunol 40, 1295-1305, doi:10.1016/j.molimm.2003.11.017 (2004).
13. Feng, J., Garrity, D., Call, M. E., Moffett, H. & Wucherpfennig, K. W. Convergence on a distinctive assembly mechanism by unrelated families of activating immune receptors.

Immunity 22, 427-438, doi:10.1016/j.immuni.2005.02.005 (2005).
14. Feng, J., Call, M. E. & Wucherpfennig, K. W. The assembly of diverse immune receptors is focused on a polar membrane-embedded interaction site. PLoS Biol 4, e142, doi:10.1371/journal.pbio.0040142 (2006).
15. Garrity, D., Call, M. E., Feng, J. & Wucherpfennig, K. W. The activating NKG2D receptor assembles in the membrane with two signaling dimers into a hexameric structure. Proc Natl Acad Sci USA 102, 7641-7646, doi:10.1073/pnas.0502439102 (2005).
16. Call, M. E., Wucherpfennig, K. W. & Chou, J. J. The structural basis for intramembrane assembly of an activating immunoreceptor complex. Nat Immunol 11, 1023-1029, doi:10.1038/ni.1943 (2010).
17. Connolly, A. & Gagnon, E. Electrostatic interactions: From immune receptor assembly to signaling. Immunol Rev 291, 26-43, doi:10.1111/imr.12769 (2019).
18. Dobbins, J. et al. Binding of the cytoplasmic domain of CD28 to the plasma membrane inhibits Lck recruitment and signaling. Sci Signal 9, ra75, doi:10.1126/scisignal.aaf0626 (2016).
19. Bettini, M. L. et al. Membrane association of the CD3epsilon signaling domain is required for optimal T cell development and function. J Immunol 193, 258-267, doi:10.4049/jimmunol.1400322 (2014).
20. Xu, C. et al. Regulation of T cell receptor activation by dynamic membrane binding of the CD3epsilon cytoplasmic tyrosine-based motif. Cell 135, 702-713, doi:10.1016/j.cell.2008.09.044 (2008).
21. Gagnon, E. et al. Response multilayered control of T cell receptor phosphorylation. Cell 142, 669-671, doi:10.1016/j.cell.2010.08.019 (2010).
22. Li, L. et al. Ionic CD3-Lck interaction regulates the initiation of T-cell receptor signaling. Proc Natl Acad Sci USA 114, E5891-E5899, doi:10.1073/pnas.1701990114 (2017).
23. Schubert, D. A. et al. Self-reactive human CD4 T cell clones form unusual immunological synapses. J Exp Med 209, 335-352, doi:10.1084/jem.20111485 (2012).
24. Gagnon, E., Schubert, D. A., Gordo, S., Chu, H. H. & Wucherpfennig, K. W. Local changes in lipid environment of TCR microclusters regulate membrane binding by the CD3epsilon cytoplasmic domain. J Exp Med 209, 2423-2439, doi:10.1084/jem.20120790 (2012).
25. Thompson, A. et al. Tandem mass tags: a novel quantification strategy for comparative analysis of complex protein mixtures by MS/MS. Anal Chem 75, 1895-1904, doi:10.1021/ac0262560 (2003).

1-92. (canceled)

93. A modular chimeric receptor comprising:
a synthetic receptor module comprising an extracellular domain fused to a first synthetic transmembrane domain;
a synthetic signaling module comprising an intracellular signaling domain fused to a second synthetic transmembrane domain;
wherein the synthetic receptor module and the synthetic signaling module form the modular chimeric receptor:
wherein the first synthetic transmembrane domain comprises:
a sequence having at least 40% identity with the sequence VLIGTSVVKLPFTILLFFL (SEQ ID NO:16),
wherein the lysine residue at position 9 of SEQ ID NO: 16 is replaced by an uncharged amino acid residue; and
(i) at least one of: the glycine residue at position 4, the threonine residue at position 5, or the serine residue at position 6 of SEQ ID NO: 16 is replaced by a positively charged amino acid; and/or
(ii) at least one of: the phenylalanine residue at position 12, the threonine residue at position 13, or the isoleucine residue at position 14 of SEQ ID NO: 16 is replaced by a positively charged amino acid;
wherein the second synthetic transmembrane domain comprises:
a sequence having at least 40% identity with the sequence VLAGIVMGDLVLTVLIALAVYFL (SEQ ID NO:26), wherein:
the aspartic acid residue at position 9 of SEQ ID NO: 26 is replaced by an uncharged amino acid residue; and
(i) at least one of: the glycine residue at position 4, the isoleucine residue at position 5, or the valine residue at position 6 is replaced by a negatively charged amino acid;
(ii) at least one of: the leucine residue at position 12, the threonine residue at position 13, or the valine residue at position 14 is replaced by a negatively charged amino acid; and/or
(iii) at least one of: the isoleucine residue at position 16, the alanine residue at position 17, or the leucine residue at position 18 is replaced by a negatively charged amino acid residue.

94. The modular chimeric receptor of claim 93, wherein the first synthetic transmembrane domain comprises a positively charged residue at position 13 in place of the threonine.

95. The modular chimeric receptor of claim 93, wherein the first synthetic transmembrane domain comprises the sequence selected from the group consisting of
(SEQ ID NO: 38)
VLILLLLLTLLLKLLLFFLL,
(SEQ ID NO: 32)
VLIGTSVVLLPFKILLFFLL,
(SEQ ID NO: 33)
VLIILLVGTSVVKLLLFFLL,
SEQ ID NO: 34)
VLIGTSVVTLPFKILLFFLL,
(SEQ ID NO: 35)
VLILLLLLLLLLKLLLFFLL,
and
(SEQ ID NO: 36)
VLILLLLGLLLLKLLLFFLL,
(SEQ ID NO: 37)
VLILLLLLALLLKLLLFFLL

96. The modular chimeric receptor of claim 93, wherein the second synthetic transmembrane domain comprises a negatively charged residue at position 13 in place of the threonine.

97. The modular chimeric receptor of claim 93, wherein the second synthetic transmembrane domain comprises the sequence of VLAGIVMGALVLDVLITLAVYFL (SEQ ID NO:39), VLALAVLGIVMGDVLITLAVYFL (SEQ ID NO:40) or VLAGDVMGTLVLIVLIALAVYFL (SEQ ID NO:41).

98. The modular chimeric receptor of claim 93, wherein the extracellular domain comprises one or more ligand-binding domains or antigen-binding domains.

99. The modular chimeric receptor of claim 93, wherein the synthetic receptor module further comprises a polypeptide linker or spacer between the extracellular domain and the first synthetic transmembrane domain.

100. The modular chimeric receptor of claim 93, wherein the synthetic receptor module further comprises an intracellular domain.

101. The modular chimeric receptor of 100, wherein the intracellular domain of the synthetic receptor module comprises a sequence of an intracellular domain of a costimulatory immune receptor.

102. The modular chimeric receptor of claim 101, wherein the costimulatory immune receptor is CD28, 4-1BB, OX40, or ICOS.

103. The modular chimeric receptor of claim 93, wherein the intracellular signaling domain of the first synthetic signaling module comprises a sequence of an intracellular domain of an immune cell signaling protein and/or of a costimulatory immune receptor.

104. The modular chimeric receptor of claim 103, wherein the intracellular signaling domain of the first synthetic signaling module comprises a sequence of the intracellular domain of DAP12 or CD3 Zeta.

105. The modular chimeric receptor of claim 98, wherein the antigen-binding domain is a scFv.

106. The modular chimeric receptor of claim 105, wherein the scFv binds to CD19.

107. The modular chimeric receptor of claim 106, wherein the scFv that binds to CD19 comprises an amino acid sequence of SEQ ID NO: 65.

108. The modular chimeric receptor of claim 104, wherein the DAP12 domain comprises an amino acid sequence of SEQ ID NO: 70.

109. The modular chimeric receptor of claim 104, wherein the CD3 Zeta domain comprises an amino acid sequence of SEQ ID NO: 69.

110. The modular chimeric receptor of claim 93, wherein:
the synthetic receptor module comprises an extracellular domain that binds to CD19, a transmembrane domain comprising an amino acid sequence of SEQ ID NO: 38, and an intracellular domain comprising a CD28 costimulatory domain; and
the synthetic signaling module comprises a transmembrane domain comprising an amino acid sequence of SEQ ID NO: 39 and a CD3 zeta intracellular domain.

111. An immune cell comprising the modular chimeric receptor of claim 93.

112. The immune cell of claim 111, wherein the immune cell is a T cell or a NK cell.
